(19) United States
(12) Patent Application Publication
(10) Pub. No.: US 2024/0159746 A1
(43) Pub. Date: May 16, 2024

(54) SYSTEM AND METHOD FOR CONDUCTING A MULTIPLEXED ASSAY

(71) Applicant: MESO SCALE TECHNOLOGIES, LLC., Rockville, MD (US)

(72) Inventors: Michael TSIONSKY, Derwood, MD (US); John KENTEN, Boyds, MD (US)

(21) Appl. No.: 18/548,444

(22) PCT Filed: Feb. 28, 2022
(86) PCT No.: PCT/US2022/018139

§ 371 Date: Aug. 30, 2023

Related U.S. Application Data
(60) Provisional application No. 63/155,008, filed on Mar. 1, 2021.

(51) Int. Cl. G01N 33/543; G01N 33/58

(52) U.S. Cl. CPC G01N 33/54306; G01N 33/582; G01N 2458/10; G01N 2458/30; G01N 2474/00

(57) ABSTRACT

Described herein are methods, systems and kits for conducting an assay for one or more analytes of interest in a sample. In one aspect, a method is provided for conducting a multiplexed binding assay for a plurality of analytes of interest in a sample. In one aspect, the assay includes an analyte binding step and a detection step. In one aspect, the analyte binding step is separated from the detection step.

### FIELD OF THE INVENTION

The present disclosure relates to assays for detecting and measuring analytes in a sample. In particular, the disclosure relates to multiplexed assays for detecting and measuring a plurality of analytes in a sample.

### BACKGROUND OF THE INVENTION

Numerous methods and systems are available for detecting and quantifying analytes of interest in biochemical and biological samples and include methods and systems for detecting and quantifying analytes such as microorganisms, pharmaceuticals, hormones, viruses, antibodies, nucleic acids and other proteins in a sample.

Many assay methods take advantage of the specificity of many biochemical and biological binding reactions using binding partners such as antigen-antibody, complementary nucleic acids, or protein-ligand binding partners. Frequently, the presence of a target analyte is indicated by the presence or absence of an observable label attached to one or more binding materials.

Electrochemiluminescent (ECL) assays provide a sensitive and precise measurement of the presence and concentration of a target analyte. Such techniques use labels or other reactants that can be induced to luminesce when electrochemically oxidized or reduced in an appropriate chemical environment. For example, electrochemiluminescence can be triggered by a voltage imposed on a working electrode at a particular time and in a particular manner. The light produced by the label is measured and indicates the presence or quantity of the analyte.

Multiplex ECL assays are available that involve the use of a multi-well plate on which an array of binding domains that include one or more binding partners of one or more analytes of interest have been immobilized. Other ECL assays involve forming a complex that includes a microparticle on which a binding partner of a target analyte has been immobilized. For multiplex assays using microparticles, the microparticles are typically uniquely labeled with fluorescent labels, see, for example, U.S. Patent Publication Nos. 2009/0163378 and 2017/0343466, which describe the use of labeled microparticles to form multidimensional arrays.

Although assays are available that provide a sensitive and precise measurement of the presence and concentration of a target analyte in a sample, there remains a need for faster and more efficient assays.

### SUMMARY OF THE INVENTION

Described herein is a method for conducting an assay for one or more analytes of interest in a sample. In one aspect, a method is provided for conducting a multiplexed assay for a plurality of analytes of interest in a sample.

In one aspect, the assay includes an analyte binding step and a detection step. In one aspect, the analyte binding step is separated from the detection step. As described herein, separating the binding step from the detection step allows for faster binding of the analyte to the detection reagent and can substantially reduce the overall time required to perform the assay.

In one aspect, the analyte binding step includes combining the sample with a detection reagent to form a binding complex that includes the detection reagent and the bound analyte. In one aspect, the detection reagent includes an analyte binding portion, a targeting reagent and a label. In one aspect, the analyte binding step includes combining the sample with a particulate support surface, a capture molecule and a detection reagent, and the binding complex is a sandwich complex that includes the particulate support surface on which a sandwich structure that includes the capture molecule, bound analyte and detection reagent is immobilized.

In one aspect, the method includes a release step. In one aspect, the binding complex is contacted with a release reagent to release a detectable portion of the binding complex. In one aspect, the detectable portion of the binding complex includes the targeting reagent and the label. In one aspect, the detectable portion of the binding complex includes a sandwich component that is released from the particulate support surface. In one aspect, the sandwich component includes labeled targeting reagent, labeled detection reagent, labeled detection reagent with bound analyte or a labeled sandwich structure.

In one aspect, the method includes a detection step. In one aspect, the released detectable portion is transferred to an assay surface on which a targeting reagent complement is immobilized. In one aspect, the released sandwich components are transferred to an assay surface on which a targeting reagent complement is immobilized. In one aspect, the detection step includes detecting the presence of the label. In one aspect, the detection step includes detecting the presence of a label immobilized on an assay surface. In one aspect, the label is a fluorescent label. In one aspect, the label is an electrochemiluminescent (ECL) label. In one aspect, the amount of label is proportional to an amount of analyte in the sample.

In one aspect, the method includes
(a) contacting the sample with a detection reagent that includes an analyte binding portion, a targeting reagent, and a label to form a binding complex that includes bound analyte and the detection reagent;
(b) removing unbound detection reagent;
(c) contacting the binding complex with a release reagent to release a detectable portion of the binding complex, wherein the detectable portion includes the targeting reagent and the label;
(d) transferring the detectable portion released from the binding complex to an assay surface on which a targeting reagent complement is immobilized under conditions in which the targeting reagent hybridizes to the targeting reagent complement; and
(e) detecting a presence of the label immobilized on the assay surface, wherein an amount of the label is proportional to an amount of analyte in the sample.

In one aspect, the sample includes an environmental or clinical sample.

In one aspect, the analyte is in suspension. In one aspect the analyte is a cell surface marker and the sample is a cell suspension. In one aspect, the analyte is not in suspension.

In one aspect, the sample includes a tissue, cell, a cell suspension, or organism. In one aspect, the sample includes a live tissue sample. In one aspect, the sample includes a fixed tissue sample. In one aspect, the analyte is associated with a tissue, cell or organism. In one aspect, the analyte includes a cell surface marker. In one aspect, the cell surface marker includes a protein, glycoprotein, enzyme or carbohydrate. In one aspect, the cell surface marker includes an integral membrane protein. In one aspect, the cell surface marker includes a transmembrane protein. In one aspect, the cell surface marker includes a peripheral membrane protein. In one aspect, the analyte binding portion of the detection reagent includes an analyte binding antibody that specifically binds an analyte or an antigen binding portion thereof.

In one aspect, sample includes a protein, polypeptide, oligonucleotide, lipid, steroid, carbohydrate, porphyrin, alkaloid, virus, microorganism, cell, tissue or subcellular particle. In one aspect, the sample is immobilized on a surface. In one aspect, the sample includes a tissue, cell, tissue, organ, organelle or organism. In one aspect, the sample includes a live tissue sample. In one aspect, the sample includes a fixed tissue sample.

In one aspect, the analyte includes an oligonucleotide. In one aspect, the analyte includes single stranded DNA, double stranded DNA, single stranded RNA or double stranded RNA. In one aspect, the analyte includes miRNA, therapeutic RNA, mRNA, or an RNA virus. In one aspect, the analyte includes a peptide. In one aspect, the analyte includes a protein. In one aspect, the analyte includes an antibody, enzyme, receptor, hormone or structural protein.

In one aspect, the analyte includes a cell surface marker. In one aspect, the cell surface marker includes a protein, glycoprotein, enzyme or carbohydrate. In one aspect, the analyte is associated with a tissue, cell or organism. In one aspect, the cell surface marker includes an integral membrane protein. In one aspect, the cell surface marker includes a transmembrane protein. In one aspect, the cell surface marker includes a peripheral membrane protein.

In one aspect, the analyte binding portion of the detection reagent is selected from an analyte binding oligonucleotide that includes an analyte binding nucleic acid sequence that is complementary to a nucleic acid sequence of the analyte, an analyte binding antibody that specifically binds an analyte, an analyte binding receptor that specifically binds an analyte that includes a ligand, or an analyte binding ligand that specifically binds an analyte that includes a receptor. In one aspect, the analyte binding portion of the detection reagent includes an analyte binding oligonucleotide that includes an analyte binding nucleic acid sequence that is complementary to a nucleic acid sequence of the analyte. In one aspect, the analyte binding portion of the detection reagent includes an analyte binding antibody or antigen binding antibody fragment that specifically binds an analyte.

In one aspect, the sample is contacted with the detection reagent, a capture molecule and a particulate support surface in (a). In one aspect, the binding complex in (a) includes a sandwich complex that includes a particulate support surface on which a sandwich structure that includes capture molecule, bound analyte and detection reagent is immobilized.

In one aspect, (c) includes contacting a sandwich complex with the release reagent and the detectable portion includes a sandwich component that is released from the particulate support surface. In one aspect, the sandwich component released from the particulate support surface includes labeled targeting reagent, labeled detection reagent, labeled detection reagent with bound analyte or a labeled sandwich structure.

In one aspect, (d) includes transferring the released sandwich component to the assay surface.

In one aspect, the analyte binding portion of the detection reagent includes an oligonucleotide. In one aspect, the analyte binding portion of the detection reagent includes a single stranded oligonucleotide. In one aspect, the analyte binding portion of the detection reagent includes an oligonucleotide with a nucleotide sequence that is complementary to a nucleotide sequence of the analyte.

In one aspect, the analyte binding portion of the detection reagent includes a protein. In one aspect, the analyte binding portion of the detection reagent includes protein selected from an antibody or receptor, or a portion thereof, that specifically binds an analyte. In one aspect, the analyte binding portion of the detection reagent includes an analyte binding antibody that specifically binds the analyte or an antigen binding antibody fragment.

In one aspect, the method includes:
(a) in one or more steps, combining the sample with a particulate support surface, a capture molecule and a detection reagent, wherein the detection reagent includes an analyte binding portion, a targeting reagent and a label, to form a sandwich complex that includes the particulate support surface on which a sandwich structure that includes capture molecule, bound analyte and detection reagent are immobilized;
(b) removing unbound detection reagent;
(c) contacting the sandwich complex with a release reagent to release a sandwich component from the particulate support surface;
(d) transferring the released sandwich component to an assay surface on which a targeting reagent complement is immobilized; and
(e) detecting a presence of the label immobilized on the assay surface, wherein an amount of label is proportional to an amount of analyte in the sample.

In one aspect, step (a) includes:
(i) combining the sample with a coated particle that includes a particulate support surface on which a capture molecule is immobilized;
(ii) allowing analytes in the sample to bind to the capture molecules to form an analyte coated particle that includes the coated particle and bound analyte;
(iii) washing the analyte coated particle to remove unbound analyte; and
(iv) contacting the washed analyte coated particle with a detection reagent that includes an analyte binding portion, a targeting reagent and a label to form a sandwich complex that includes the particulate support surface and sandwich components.

In one aspect, step (a) includes:
(i) combining the sample with a capture molecule and a detection reagent;
(ii) allowing analytes in the sample to bind to the capture molecules and detection reagents to form a sandwich structure that includes the capture molecule, the analyte and the detection reagent; and
(iii) contacting a particulate support surface with the sandwich structure and immobilizing the sandwich structure on the particulate support to form a sandwich complex.

In one aspect, the capture molecule includes an immobilization reagent.

In one aspect, the particulate support surface is coated with avidin or streptavidin and the immobilization reagent includes biotin.

In one aspect, the particulate support surface includes a magnetic particle.

In one aspect, the capture molecule immobilized on the particulate support surface is selected from: a capture oligonucleotide with a capture nucleic acid sequence that is complementary to a first nucleic acid sequence of the analyte, a capture antibody that specifically binds a first portion of an analyte, a capture receptor that specifically binds a first portion of an analyte that includes a ligand, or a capture ligand that specifically binds a first analyte that includes a receptor.

In one aspect, each particulate support surface includes capture molecules specific for one analyte. In one aspect, each particulate support surface includes two or more different capture molecules specific for different analytes. In one aspect, each particulate support surface includes a plurality of capture molecules specific for a plurality analytes.

In one aspect, the targeting reagent includes a single stranded oligonucleotide sequence. In one aspect, the targeting reagent includes a nucleotide sequence that is complementary to a nucleotide sequence of the targeting reagent complement.

In one aspect, the targeting reagent is attached to the analyte binding portion of the detection reagent via a digestible linker. In one aspect, the digestible linker includes a disulfide bond, a short peptide sequence or a protein. In one aspect, the digestible linker includes a nucleic acid sequence. In one aspect, the digestible linker includes a restriction site.

In one aspect, the release agent includes a protease. In one aspect, the protease is selected from: a serine protease, cysteine protease, threonine protease, aspartic protease, glutamic protease, and metallo protease. In one aspect, the protease includes a serine protease. In one aspect, the protease includes proteinase K.

In one aspect, the release reagent includes a reducing reagent and the digestible linker includes a disulfide bond. In one aspect, the reducing agent is β-mercaptoethanol (BME) or dithiothritol (DTT). In one aspect, the digestible linker includes a nucleic acid sequence that includes a restriction site and the release reagent includes a restriction enzyme.

In one aspect, one or more of the capture molecule, the detection reagent, the analyte binding portion of the detection reagent, or the targeting reagent includes a nucleotide sequence that includes a restriction site.

In one aspect, the assay surface includes a carbon-based electrode. In one aspect, the carbon-based electrode includes a carbon ink electrode. In one aspect, a targeting reagent complement is immobilized on a binding domain on the assay surface. In one aspect, the assay surface includes a multi-well plate. In one aspect, the multi-well plate includes a carbon-based electrode. In one aspect, the multi-well plate includes an electrode in a well of the multi-well plate. In one aspect, a targeting reagent complements is immobilized on a binding domain on an electrode in a well of the multi-well plate.

In one aspect, the label includes an electrochemiluminescent label. In one aspect, the label includes an organometallic complex that includes a transition metal. In one aspect, the transition metal includes ruthenium. In one aspect, the label includes a MSD SULFO-TAG™ label (available from Meso Scale Discovery LLC, Rockville, MD).

In one aspect, the method includes amplifying the detectable portion after the detectable portion is released from the binding complex. In one aspect, amplifying the detectable portion includes PCR amplification of an oligonucleotide sequence of the targeting reagent.

In one aspect, the label includes a primer for rolling circle amplification (RCA primer). In one aspect, detecting the presence of the label immobilized on the assay surface includes contacting the assay surface with a template for rolling circle amplification (RCA template) and generating a rolling circle amplification product (RCA product). In one aspect, detecting the presence of the label immobilized on the assay surface includes contacting the RCA product with a detection probe. In one aspect, the detection probe includes a label and a nucleotide sequence that is complementary to a nucleotide sequence of the RCA product.

In one aspect, the label includes a fluorescent label. In one aspect, the detection probe includes more than one type of fluorescent label. In one aspect, the detection probe has a fluorescent signature based on a ratio of the fluorescent labels on the detection probe.

In one aspect, the detection reagent is conjugated to a carrier protein that includes more than one labeled targeting reagent. In one aspect, the carrier protein is digested with a digestion reagent after the sandwich components are released from the particulate support surface to release the labeled targeting reagent. In one aspect, the digestion reagent includes an enzyme. In one aspect, the digestion reagent includes a protease. In one aspect, the labeled targeting reagent is conjugated to the carrier protein through a disulfide bond. In one aspect, the digestion reagent includes a reducing agent. In one aspect, the reducing agent is selected from β-mercaptoethanol (BME) and dithiothritol (DTT). In one aspect, the labeled targeting reagent is conjugated to the carrier protein through an oligonucleotide that includes a restriction site.

In one aspect, the method includes:
(a) contacting the sample with a detection reagent that includes an analyte binding portion, a targeting reagent, and a label that includes a primer for rolling circle amplification (RCA primer), wherein a binding complex is formed that includes bound analyte and the detection reagent;
(b) removing unbound detection reagent;
(c) contacting the binding complex with a release reagent to release a detectable portion of the binding complex, wherein the detectable portion includes the targeting reagent and the label;
(d) transferring the detectable portion released from the binding complex to an assay surface on which a targeting reagent complement is immobilized under conditions in which the targeting reagent hybridizes to the targeting reagent complement;
(e) contacting the assay surface with a template for rolling circle amplification (RCA template) under conditions in which a rolling circle amplification product (RCA product) is generated;
(f) contacting the RCA product with a labeled detection probe that includes a nucleotide sequence that is complementary to a sequence of the RCA product; and
(g) detecting presence of the labeled detection probe immobilized on the assay surface.

In one aspect, the sample includes more than one target analyte. In one aspect, the method includes contacting the sample with a unique detection reagent for each target analyte in the sample. In one aspect, each unique detection reagent includes a unique analyte binding portion.

In one aspect, each unique detection reagent includes a unique RCA primer. In one aspect, the assay surface is contacted with a unique RCA template corresponding to each unique RCA primer to form a unique RCA product. In one aspect, each unique RCA product is contacted with a unique labeled detection probe having a unique fluorescent signature. In one aspect, each labeled detection probe includes a unique fluorescent label. In one aspect, each labeled detection probe includes a unique ratio of fluorescent labels.

In one aspect, a system or kit for conducting an assay to detect an analyte in a sample is provided. In one aspect, the kit includes:
(a) an analyte binding system that includes a detection reagent that includes an analyte binding portion, a targeting reagent and a detectable label;
(b) a release reagent; and
(c) a detection system that includes an assay surface and a targeting reagent complement.

In one aspect, the system or kit further includes a particulate support surface and a capture molecule.

In one aspect, the particulate support surface in the system or kit includes a magnetic or paramagnetic particle. In one aspect, the particulate support surface in the system or kit is coated with an affinity binding ligand. In one aspect, the affinity binding ligand is selected from avidin, streptavidin and biotin. In one aspect, the particulate support surface includes a carboxyl or amine coating.

In one aspect, the system or kit further includes an immobilization reagent for immobilizing a capture molecule on the particulate support surface. In one aspect, the immobilization reagent is selected from avidin, streptavidin and biotin.

In one aspect, the kit includes an assay surface. In one aspect, the assay surface in the system or kit includes a multi-well plate. In one aspect, the assay surface includes a plurality of particles. In one aspect, the assay surface includes a plurality of discrete binding domains. In one aspect, the assay surface includes a targeting reagent complement immobilized thereon. In one aspect, the assay surface includes more than one unique targeting reagent complements immobilized in one or more discrete binding domains. In one aspect, the assay surface includes a plurality of targeting reagent complements immobilized in a plurality of discrete binding domains.

In one aspect, the system or kit includes, in a separate vial, container, or compartment, one or more of the following:
(a) a targeting reagent complement that can be immobilized on the assay surface;
(b) a detection reagent or one or more components of a detection reagent; or
(c) a labeled targeting reagent.

In one aspect, the system or kit includes a labeled targeting reagent. In one aspect, the label includes an electrochemiluminescent (ECL) label. In one aspect, the label includes an organometallic complex that includes a transition metal. In one aspect, the label includes ruthenium. In one aspect, the label includes a MSD SULFO-TAC™ label.

In one aspect, the labeled targeting reagent includes a primer for rolling circle amplification (RCA primer). In one aspect, the system or kit includes a template for rolling circle amplification (RCA template). In one aspect, the system or kit includes a detection probe that is capable of hybridizing to a rolling circle product (RCA product) generated using the RCA template.

In one aspect, the system or kit includes a detection probe that includes a label. In one aspect, the detection probe includes a fluorescent label.

In one aspect, the targeting reagent in the system or kit includes an oligonucleotide sequence that is complementary to an oligonucleotide sequence of a targeting reagent complement.

In one aspect, the system or kit includes a release reagent. In one aspect, the release reagent is selected from a protease, an endopeptidase, a reducing reagent and a restriction enzyme. In one aspect, the protease is selected from: a serine protease, cysteine protease, threonine protease, aspartic protease, glutamic protease, and metalloprotease. In one aspect, the protease includes proteinase K. In one aspect, the reducing agent is an endonuclease. In one aspect, the release agent is a reducing agent.

### BRIEF DESCRIPTION OF THE FIGURES

FIG. 1A is a schematic of a detection reagent described herein.

FIG. 1B is a schematic of a sandwich structure described herein.

FIG. 1C is a schematic of a sandwich complex described herein.

FIG. 1D is a schematic of an alternate detection reagent described herein.

FIG. 1E is a schematic of an alternate sandwich structure described herein

FIG. 2 is a schematic showing analyte binding to capture molecules immobilized on a support surface wherein more than one different capture molecules specific for more than one different target analytes are immobilized on the same support surface.

FIG. 3 is a schematic showing analyte binding to capture molecules immobilized on a support surface wherein different capture molecules specific for different target analytes are immobilized on separate support surfaces.

FIG. 4 is a schematic showing an analyte binding step in which a sample is contacted with one or more capture molecules and detection reagents to form sandwich complexes which are then immobilized on a support surface.

FIG. 5 is a schematic showing exemplary sandwich components that may be released after a sandwich complex described herein is contacted with a release agent.

FIG. 6A is a schematic showing a detection reagent that includes a digestible linker.

FIG. 6B is a schematic showing the release of sandwich components from a sandwich complex that includes digestible linkers.

FIG. 7 is a schematic of a binding complex described herein.

FIG. 8 is a flow chart providing an overview of an assay described herein.

FIG. 9 is a flow chart providing an overview of an alternate assay described herein.

FIG. 10 is a flow chart providing an overview of an alternate assay described herein.

FIG. 11 is a schematic of a detection method described herein.

FIG. 12 is a schematic of an alternate detection method described herein.

FIG. 13 is a schematic of an assay in which the detection reagent includes an amplification primer for rolling circle amplification as a label.

FIG. 14 is a schematic of a multiplex assay in which the detection reagent includes an amplification primer for rolling circle amplification as a label.

### DETAILED DESCRIPTION OF THE INVENTION

### A. Definitions

Unless otherwise defined, scientific and technical terms used herein shall have the meanings that are commonly understood by those of ordinary skill in the art. Further, unless otherwise required by context, singular terms shall include pluralities and plural terms shall include the singular, for example, “a” or “an”, include pluralities, e.g., “one or more” or “at least one” and the term “or” can mean “and/or”, unless stated otherwise. The terms “including,” “includes” and “included”, are not limiting. Ranges provided herein, of any type, include all values within a particular range described and values about an endpoint for a particular range.

As used herein, the term “about” is used to modify, for example, the quantity of an ingredient in a composition, concentration, volume, process temperature, process time, yield, flow rate, pressure, and ranges thereof, employed in describing the invention. The term “about” refers to variation in the numerical quantity that can occur, for example, through typical measuring and handling procedures used for making compounds, compositions, concentrates or formulations; through inadvertent error in these procedures; through differences in the manufacture, source, or purity of starting materials or ingredients used to carry out the methods, and other similar considerations. The term “about” also encompasses amounts that differ due to aging of a composition. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation. Where modified by the term “about,” the claims appended hereto include such equivalents.

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Binding pair” refers to two molecules that specifically bind to each other. Examples of binding pairs include, but are not limited to, complementary oligonucleotides; antibody-antigen binding pairs; receptor-ligand binding pairs; enzyme-substrate binding pairs; hapten and hapten binding partner; and biotin and streptavidin or avidin. In one aspect, the binding pair includes an oligonucleotide sequence and a corresponding complementary oligonucleotide sequence that do not bind or cross-react with other oligonucleotide sequences present in a sample, composition or mixture under stringent conditions. In one aspect, the binding pair include an antigen-antibody pair or a receptor-ligand pair.

The term “specifically binds” means that a first member of a binding pair has a higher affinity for a second member of the binding pair as compared to other components in a sample. In one aspect, one member of a binding pair binds to the other member of the binding pair under suitable conditions without any significant binding, for example, without any statistically significant binding, to other components present in a sample. In one aspect, members of a binding pair have an affinity for each other that is at least about 50-, 75-, or 100-fold greater than the affinity between either member of the binding pair and other components present in the sample. In one aspect, the term “specifically binds” means that a capture molecule has a higher affinity for a particular target analyte in a sample as compared to other components in the sample, including other analytes in the sample. In one aspect, the term “specifically binds” means that an analyte binding portion of a detection reagent has a higher affinity for a target analyte in a sample as compared to other components in the sample, including other analytes in the sample. In one aspect, the term “specifically binds” means that a targeting reagent has a higher affinity for its corresponding targeting reagent complement as compared to other targeting reagent complements immobilized on an assay surface.

As used herein, the term “corresponding” refers to the relationship between members of a binding pair, for example, a capture molecule and an analyte, an analyte binding portion of a detection reagent and an analyte, or a targeting reagent and a targeting reagent complement. In one aspect, a member of a binding pair specifically binds to the “corresponding” member of the binding pair under suitable conditions without any significant binding, for example, without any statistically significant binding, to other components present in a sample or assay.

“Unique” is a relative term that depends on the other components present in a composition or mixture. For example, when used in connection with a nucleotide sequence, for example the nucleotide sequence of an analyte binding portion of a detection reagent, the term “unique” means that the nucleotide sequence of one analyte binding portion is different from the nucleotide sequence of the analyte binding portion of the other detection reagents in the composition or mixture. When used in connection with a protein, such as an antibody, for example an analyte binding portion of a detection reagent that includes an antibody, the term “unique” means that the antibody of one analyte binding portion specifically binds to a different target analyte than a different analyte binding portion of the other detection reagents in the composition or mixture. Similarly, when used in connection with a targeting reagent, the term “unique” means that the nucleotide sequence of the targeting reagent is different from the nucleotide sequence of other targeting reagents in the composition or mixture or that the nucleotides sequence of a targeting reagent complement is different from the nucleotide sequence of other targeting reagent complements in the composition or mixture. Alternately, the term “unique” means that a peptide sequence, or an antibody of one targeting reagent specifically binds to a different target analyte than a different analyte binding portion of the other detection reagents in the composition or mixture. The term “unique” does not preclude the possibility that multiple copies of a “unique” analyte or reagent may be present in an assay or sample. When used in connection with a label, the term “unique” means that the label has a detectable signal that is distinguishable from other labels in the composition or mixture. For a fluorescent label, a unique label is a label that has a spectral characteristic that distinguishes it from other fluorescent labels in the same composition or mixture. For example, a fluorescein label can be a unique label if it is in a mixture containing a rhodamine label. In one aspect, the label includes more than one fluorescent moiety and the unique spectral signature due to the ratio of the fluorescent moieties renders the label distinguishable from other labels in the composition or mixture.

In the context of analytes measured in an assay, or a reagents used in an assay, the term “plurality” means more than one structurally and/or functionally different analyte or reagent (e.g., reagent A and reagent B), rather than just more than one copy of the analyte or reagent (e.g., reagent A and another copy of reagent A). For example, the term “plurality of detection reagents” means that more than one structurally or functionally different detection reagent is present in an assay, for example, the different detection reagents each specifically bind a different target analyte and does not describe a situation where there are multiple copies of one reagent. However, use of the term “plurality” in this context does not preclude the possibility that multiple copies are present of any of the plurality of analytes or reagents. For example, a plurality of immobilized targeting reagent complements could refer to immobilized targeting reagent complements that include one or more copies of targeting reagent complement A and one or more copies of targeting reagent complement B. When referring to a plurality of analytes or reagents, the terms “first,” “second,” “third,” etc. or “additional” can be used to distinguish between the unique analytes or reagents. For example, a “first” detection reagent binds to a “first” target analyte and a “second” detection reagent binds to a “second” target analyte or a different portion of the target analyte.

“Complementary” refers to nucleic acid molecules or a sequence of nucleic acid molecules that interact by the formation of hydrogen bonds, for example, according to the Watson-Crick base-pairing model, for example, in which A pairs with T or U; and C pairs with G. For example, hybridization can occur between two complementary DNA molecules (DNA-DNA hybridization), two complementary RNA molecules (RNA-RNA hybridization), or between complementary DNA and RNA molecules (DNA-RNA hybridization). Perfect complementarity or 100% complementarity refers to a situation in which each nucleotide of one oligonucleotide sequence or region can hydrogen bond with each nucleotide of at least a portion of consecutive nucleotides of a second oligonucleotide strand or region. Hybridization can occur between sequences that do not have 100% sequence complementarity (i.e., sequences where less than 100% of the nucleotides align based on a base-pairing model such as the Watson-Crick base-pairing model). Generally, sequences having less sequence complementarity are less stable and less likely hybridize than sequences having greater sequence complementarity. In one aspect, the nucleotides of the complementary sequences have 100% sequence complementarity based on the Watson-Crick model. In another aspect, the nucleotides of the complementary sequences have at least about 90%, 95%, 96%, 97%, 98% or 99% sequence complementarity along at least a portion of consecutive nucleotides based on the Watson-Crick model and are able to hybridize under stringent hybridization conditions. In one aspect, the complementary oligonucleotide sequences hybridize along their entire length. However, the complementary sequences need not hybridize along their entire length. In one aspect, a shorter oligonucleotide sequence can hybridize to a portion of a longer oligonucleotide sequence to which is it complementary.

Whether or not two complementary sequences hybridize can depend on the stringency of the hybridization conditions, which can vary depending on conditions such as temperature, solvent, ionic strength and other parameters. The stringency of the hybridization conditions can be selected to provide selective formation or maintenance of a desired hybridization product of two complementary nucleic acid sequences, in the presence of other potentially cross-reacting or interfering sequences. Stringent conditions are sequence-dependent—typically longer complementary sequences selectively hybridize at higher temperatures than shorter complementary sequences.

The term “identical” means that two polynucleotide or two polypeptide sequences include identical nucleic acid bases or identical amino acid residues, respectively, at the same positions over a comparison window. “% sequence identity” can be determined by comparing two aligned sequences over a window of comparison, determining the number of positions at which the identical nucleic acid base or amino acid residue occurs in both sequences to yield the number of matched positions, dividing the number of matched positions by the total number of positions in the comparison window, and multiplying the result by 100 to yield the percentage of sequence identity. The comparison window can include a full-length sequence or may be a subpart of a larger sequence. Various methods and algorithms are known for determining the percent identity between two or sequences, including, but not limited MEGALIGN (DNASTAR, Inc. Madison, Wis.), FASTA, BLAST, or ENTREZ.

The term “oligonucleotide” refers to a short polymer of nucleic acids with a phosphate backbone, and includes, but is not limited to, deoxyribonucleic acid (DNA), ribonucleic acid (RNA), locked nucleic acid (LNA), peptide nucleic acid (PNA), or combinations thereof. In one aspect, the oligonucleotide has a length from about 5 to about 150 nucleotides. An oligonucleotide may be single-stranded or double-stranded and may be obtained by methods, including, but not limited to, isolation from a biological sample, recombinant synthesis and chemical synthesis. The term “oligonucleotide” may include non-naturally occurring nucleotide bases or modifications. For example, an oligonucleotide may include a chemical modification that links it to another substance such as a label or provides a reactive functional group that can be linked to another substance. For example, the oligonucleotide can include Iso-dC and/or Iso-dG, which are chemical variants of cytosine and guanine, respectively, available from EraGen Biosciences, Inc. (www.eragen.com). Incorporation of such modified bases into oligonucleotide sequences effectively expands the genetic alphabet and permits synthesis of oligonucleotides that have increased specificity and decreased mismatch hybridization potential.

Oligonucleotide sequences and complementary sequences can be generated by techniques known in the art for generating pairs of complementary oligonucleotides with similar binding energies (or melting temperatures) and low inter-pair cross-reactivity (e.g., commercial or public software for selecting probes or primers for multiplexed nucleic acid assays). Oligonucleotide sequences that can be used for targeting reagent-targeting reagent complement binding pairs are disclosed, for example, in U.S. Patent Publication No. 2016/0069872 (application Ser. No. 14/847,761, filed Sep. 8, 2015, entitled “METHODS FOR CONDUCTING MULTIPLEXED ASSAYS”), the disclosure of which are hereby incorporated by reference herein in its entirety.

As used herein, “polypeptide” refers to a molecule made up of amino acid monomers linked by peptide bonds. The term polypeptide refers to any chain or chains of amino acids with a peptide backbone and does not refer to a chain of amino acids having a specific length and can include, but is not limited to, peptides, dipeptides, tripeptides, oligopeptides, and proteins. The term polypeptide also includes products of post-expression modifications of the polypeptide, including, but not limited to, glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology. A polypeptide is not necessarily translated from a designated nucleic acid sequence. It may be generated in any manner, including by chemical synthesis. In the context of polypeptides, a “sequence” refers to an order of amino acids in a polypeptide in an amino to carboxyl terminal direction in which residues that neighbor each other in the sequence are contiguous in the primary structure of the polypeptide.

The term “antibody” refers to a biomolecule that is capable of specifically binding to an antigen. In most vertebrate animals, antibodies exist as dimers of two heavy (H) chains that are each paired with a light (L) chain. The N-termini of the heavy and light chains include a variable domain (V_{H} and V_{L}, respectively) that provides the antibody with its unique antigen-binding specificity. The term “antibody” can refer to a whole antibody molecule or an antigen-binding fragment thereof. The antibody or a fragment thereof can be naturally produced, or partially or wholly synthetically or recombinantly produced. An antigen-binding fragment refers to any antibody fragment that includes at least a portion of the variable region of the immunoglobulin molecule and retains the binding specificity of the full-length immunoglobulin. The term antibody includes synthetic antibodies, recombinantly produced antibodies, multispecific antibodies (e.g., bispecific antibodies), human antibodies, non-human antibodies, humanized antibodies, chimeric antibodies, intrabodies, and antibody fragments, including, but not limited to, Fab fragments, Fab′ fragments, F(ab′)₂ fragments, FIT fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above.

“Label” refers to a chemical group or moiety that has a detectable physical property or is capable of causing a chemical group or moiety to exhibit a detectable physical property. A label can be detected by spectroscopic, photochemical, biochemical, immunochemical, electrical, optical, chemical, or other methods. Examples of labels include, but are not limited to, radioisotopes, enzymes, substrates, fluorescent molecules, chemiluminescent moieties, electrochemiluminescent moieties, magnetic particles, and bioluminescent moieties. In one aspect, the label is a compound that is a member of a binding pair, in which a first member of the binding pair (which can be referred to as a “primary binding reagent”) is attached to a substrate, for example, an oligonucleotide, and the other member of the binding pair (which can be referred to as a “secondary binding reagent”) has a detectable physical property. Non-limiting examples of binding pairs include biotin and streptavidin, or avidin; complementary oligonucleotides; hapten and hapten binding partner; and antibody-antigen binding pairs.

“Detection” refers to detecting, observing, or quantifying the presence of a substance, based on the presence or absence of a label.

A “support surface” refers to a surface material onto which a substance, for example, an oligonucleotide, polypeptide, cell or tissue can be immobilized. A “support surface” can be planar or non-planar. In one aspect, the support surface includes a flat surface. In one aspect, the support surface includes a slide, cartridge, bead, or chip. In one aspect, the support surface includes an assay surface. As used herein “assay surface” refers to a support surface on which a detectable label can be immobilized and detected. In one aspect, the support surface is a plate with more than one well, i.e., a “multi-well plate.” Multi-well plates can include any number of wells of any size or shape, arranged in any pattern or configuration. In another aspect, the support surface has a curved surface. In one aspect, the support surface is provided by one or more particles, beads or microspheres. The terms particles, beads or microspheres can be used interchangeably unless otherwise indicated. In one aspect, the support surface includes color coded particles, beads or microspheres. In one aspect, the support surface includes assay flow cells or assay fluidics. In one aspect, a support surface is used in an analyte binding step. In one aspect, a support surface is used in a detection step. In one aspect, a support surface used in a detection step is referred to as an assay surface. In one aspect, the support surface used in the analyte binding step is different from the support surface used in the detection step. In one aspect, a first support surface, for example, a particulate support surface, a surface of a culture plate or plate well, or a glass slide, is used in an analyte binding step and a second support surface, for example, an assay surface such as a multi-well plate, is used in a detection step.

“Array” refers to a support surface, for example, an assay surface, having more than one spatially distinct (i.e., not overlapping) addressable locations, referred to herein as binding domains. In one aspect, each addressable location or binding domain includes a targeting reagent complement. In one aspect, the assay surface includes a plurality of binding domains that each include a unique targeting reagent complement.

### B. Overview

Described herein are methods, systems and kits for conducting an assay for detecting one or more analytes in a sample. In one aspect, a method, system or kit is provided for a multiplexed assay for detecting a plurality of analytes in a sample. In one aspect, the assay includes an analyte binding step and a detection step. In one aspect, the analyte binding step is separated from the detection step. In one aspect, the analyte binding step and the detection step are both performed on support surfaces. In one aspect, “separated” means that the analyte binding step is performed using a different support surface than the detection step. In one aspect, “separated” means that the analyte binding step is performed at a different time than the detection step. In one aspect, “separated” means that the analyte binding step is performed using a different support surface and at a different time than the detection step.

One advantage of the method and system described herein is that the analyte binding step and the detection step require substantially less time to complete as compared to a traditional multiplex assay. In one aspect, the analyte binding step and the detection step each take less than about 1 hour, 45 minutes, 40 minutes, 35 minutes or 30 minutes, which can reduce total assay time to less than about 2 hours, 1.5 hours, or 1 hour.

In one aspect, the particulate support surface does not need to be individually labeled, for example, using different colors or wavelengths for use in a multiplex assay. In one aspect, the same support surface on which targeting reagent complements are immobilized can be used to detect a variety of different analytes by changing the analyte binding portion included in the detection reagent.

In one aspect, the analyte binding step includes combining the sample with a detection reagent to form a binding complex that includes the detection reagent and the bound analyte. In one aspect, the detection reagent includes an analyte binding portion, a targeting reagent and a label. In one aspect, the analyte binding step includes combining the sample with a particulate support surface, a capture molecule and a detection reagent, and the binding complex is a sandwich complex that includes the particulate support surface on which a sandwich structure that includes the capture molecule, bound analyte and detection reagent is immobilized.

In one aspect, the method includes a release step. In one aspect, the binding complex is contacted with a release reagent to release a detectable portion of the binding complex. In one aspect, the detectable portion of the binding complex includes the targeting reagent and the label. In one aspect, the detectable portion of the binding complex includes a sandwich component that is released from the particulate support surface. In one aspect, the sandwich component includes labeled targeting reagent, labeled detection reagent, labeled detection reagent with bound analyte or a labeled sandwich structure.

In one aspect, the method includes a detection step. In one aspect, the released detectable portion is transferred to an assay surface on which a targeting reagent complement is immobilized. In one aspect, the released sandwich component is transferred to an assay surface on which a targeting reagent complement is immobilized. In one aspect, the detection step includes detecting the presence of a label. In one aspect, the detection step includes detecting the presence of the label immobilized on an assay surface. In one aspect, the label is a fluorescent label. In one aspect, the label is an electrochemiluminescent (ECL) label. In one aspect, the amount of label is proportional to an amount of analyte in the sample.

In one aspect, the method includes contacting the sample with a detection reagent that includes an analyte binding portion, a targeting reagent, and a label to form a binding complex that includes bound analyte and the detection reagent. In one aspect, the method includes removing unbound detection reagent. In one aspect, unbound detection reagent is removed by one or more washes. In one aspect, the method includes contacting the binding complex with a release reagent to release a detectable portion of the binding complex, wherein the detectable portion includes the targeting reagent and the label. In one aspect, the method includes transferring the detectable portion released from the binding complex to an assay surface on which a targeting reagent complement is immobilized under conditions in which the targeting reagent hybridizes to the targeting reagent complement. In one aspect, the method includes detecting a presence of the label immobilized on the assay surface, wherein an amount of the label is proportional to an amount of analyte in the sample.

In one aspect, the method includes, in one or more steps, combining the sample with a particulate support surface, a capture molecule and a labeled detection reagent to form a sandwich complex that includes the particulate support surface on which a sandwich structure is immobilized. In one aspect, the method includes washing the sandwich complex to remove unbound analyte and unbound detection reagent. In one aspect, the method includes contacting the sandwich complex with a release reagent to release sandwich components from the particulate support surface. In one aspect, the method includes transferring the released sandwich components to an assay surface. In one aspect, the method includes detecting presence of the label, wherein an amount of label is proportional to an amount of analyte in the sample.

### C. Sample

The assay described herein is suitable for detecting a target analyte in a sample. As used herein, an “analyte” or “target analyte” can include any molecule of interest capable of being detected and analyzed by the methods and kits described herein. The term “target analyte” can refer to the entire molecule of interest or a segment or portion of the molecule of interest. In one aspect, the target analyte includes modified molecules, for example, labeled, cleaved, or chemically or enzymatically treated versions of a molecule of interest. In one aspect, the sample includes a clinical or environmental sample. In one aspect, the sample is a biological sample. In one aspect, the amount of a target analyte in a sample may be indicative of a disease or disease condition. In another aspect, the amount of a target analyte in a sample may indicate whether the patient was exposed to that analyte.

In one aspect, the method described herein is used to detect the presence of or quantify more than one, for example, two or more analytes in a sample. In one aspect, the method described herein is used to detect the presence of or quantify a plurality of analytes in a sample. In one aspect, the method is used to measure a panel of analytes in the same sample including, for example, a panel of analytes associated with a disease state or physiological condition.

In one aspect, the sample is a suspension. In one aspect, the sample is a cell suspension. In one aspect, the target analyte is in a suspension. In one aspect the target analyte is a cell surface marker and the sample is a cell suspension. In one aspect, the target analyte is not in suspension. As used herein, “suspension” refers to a heterogeneous mixture in which a solid component, for example, a cell, cell fragment, polypeptide or a target analyte is dispersed throughout a medium, for example, a liquid medium. The term “suspension” does not require that the solid component be distributed evenly throughout the medium. For example, the solid component may be disposed near a support surface, for example, due to the force of gravity. In one aspect, the sample includes a target analyte in a suspension, wherein the target analyte is not immobilized on a surface. In one aspect, the sample includes a solid component in suspension, wherein the target analyte is associated with or immobilized on the solid component, for example, a sample that includes a suspension of particulate support surfaces on which a target analyte is immobilized or a suspension of cells with which the target analyte is associated. In one aspect, the target analyte is immobilized on a surface. As used herein, “immobilized” refers to a state in which an analyte is affixed at an approximately constant position relative to a surface. The analyte may be attached to the surface directly or indirectly and the immobilization can be reversible or irreversible. In one aspect, the immobilized target analyte is associated with a cell. In one aspect, the immobilized target analyte is associated with an isolated cell. As used herein, “isolated cell” refers to a cell that is substantially free from other cells with which the cell is typically found in its native stale. The phrase “isolated cell” can refer to a cell in a cell culture or in a suspension of isolated cells. In one aspect, the immobilized target analyte is associated with a multicellular structure, for example, a tissue, organ, organelle or organism. In one aspect, the immobilized target analyte is associated with an isolated cell or multicellular structure that is adhered to a surface, for example, an adherent cell culture or a fixed tissue section. In one aspect, the immobilized target analyte is associated with a live cell or tissue. As used herein “associated with,” refers to a physical connection between two or more elements, for example, between a target analyte and a cell or tissue, in which the elements remain physically connected under the conditions of use, for example, under conditions in which the assay performed or physiological conditions. The physical connection can be a result of covalent or non-covalent interactions or bonds and includes direct connections and indirect connections, for example, in which the elements are connected via a linking agent.

Examples of samples that may be analyzed by the method described herein include, but are not limited to, food samples (including, but not limited to, food extracts, food homogenates, and beverages), environmental samples (including, but not limited to, soil samples, environmental sludges, collected environmental aerosols, environmental wipes, and water filtrates), industrial samples (including, but not limited to, starting materials, products or intermediates from an industrial production process), human clinical samples, veterinary samples and other samples of biological origin. Biological samples that may be analyzed include, but are not limited to, physiological fluids and/or samples containing suspensions of cells, as well as tissues and cells that are not in suspension. Specific examples of biological samples include, but are not limited to, blood, serum, plasma, feces, mucosal swabs, tissue aspirates, tissue homogenates, cell cultures and cell culture supernatants (including cultures of eukaryotic and prokaryotic cells), urine, saliva, sputum, and cerebrospinal fluid. Examples of tissues and cells that are not in suspension include, but are not limited to, fixed tissue samples, live tissue samples, perfused organs, and whole organisms. In one aspect, the method may be used to detect pathogenic and/or potentially pathogenic virus, bacteria and toxins including biological warfare agents (“BWAs”) in a clinical or environmental sample.

The target analyte can include, but is not limited to, proteins (including, for example, oligopeptides, polypeptides, glycoproteins, lipoproteins and peptide analogs), nucleic acids (including, for example, mononucleotides, oligonucleotides, polynucleotides, ribonucleic acids, deoxyribonucleic acids, and nucleic acid analogs), lipids, steroids, carbohydrates (including, for example, sugars and polysaccharides), porphyrins, alkaloids, nucleotides, nucleosides, amino acids, fatty acids, viruses, microorganisms, biological cells (including, for example, prokaryotic and eukaryotic cells), tissues, organs, or organelles.

In one aspect, the target analyte includes a target nucleotide sequence. In one aspect, the target analyte includes DNA. In one aspect, the target analyte includes RNA. In one aspect, the target analyte is single stranded. In one aspect, the target analyte is double stranded. The target nucleotide sequence can include, but is not limited to, sequences found in the DNA or RNA of prokaryotic or eukaryotic organisms. In one aspect, the target analyte includes miRNA, a therapeutic RNA, mRNA, or an RNA virus. In one aspect, the target is extracted from a sample, e.g., nuclear DNA or viral genomic DNA or RNA. In one aspect, the target is detected in a biological sample, for example, cell free fetal DNA or cell free tumor DNA in serum or plasma or a therapeutic oligonucleotide in circulation. In one aspect, the target nucleotide includes amplified sequences from a biological sample. Amplification methods are known and include, but are not limited to, polymerase chain reaction (PCR), whole genome amplification (WGA), reverse transcription followed by the polymerase chain reaction (RT-PCR), strand displacement amplification (SDA), or rolling circle amplification (RCA). Polymerases suitable for the amplification methods herein include, e.g., Taq, Phi, Bst, and Vent-exo, e.g., for DNA amplification, and T7 RNA polymerase, e.g., for RNA amplification.

In one aspect, the target analyte is a protein. In one aspect, the target analyte is a naturally occurring protein. In one aspect, the target analyte is a recombinantly produced protein. In one aspect, the target analyte is a protein that includes a single polypeptide chain. In one aspect, the target analyte is a protein that includes more than one polypeptide chain. In one aspect, the target analyte is a protein such as an antibody, enzyme, receptor, hormone or structural protein.

In one aspect, the target analyte includes a cell surface marker. As used herein, “cell surface marker” refers to a molecule, such as a protein, glycoprotein, enzyme or carbohydrate, expressed on the surface of a cell. In one aspect, a “cell surface maker” is a protein, glycoprotein, enzyme or carbohydrate that is associated with an isolated cell or multicellular structure, such as a tissue, organ, organelle or organism. In one aspect, the cell surface marker is an integral membrane protein that has one or more segments that are embedded in the lipid bilayer of the plasma membrane. In one aspect, the cell surface marker is a transmembrane protein that contains one or more membrane spanning domains. In one aspect, the cell surface marker is a peripheral membrane protein that is indirectly associated with the plasma membrane, for example, by interactions with an integral membrane protein or by interactions with lipid groups of the plasma membrane.

In one aspect, the target analyte is a cell surface marker relating to immuno-oncology. In one aspect, the target analyte is a cell surface marker that can be used to characterize T-cell activation (See, for example, Bjoern et al. (2017) “Influence of ipilimumab on expanded tumour derived T cells from patients with metastatic melanoma” Oncotarget. 8(16):27062-27074). In one aspect, the cell surface marker relates to the activity of one or more check point inhibitors for tumor infiltrating lymphocytes. In one aspect, the sample includes a population of CD4 or CD8 T-cells and the assay is used to determine the levels of one or more of the following: CD28, CD27, CTLA-4, CTLA-4 total, CD57, LAG3, PD-1, ICOS, BTLA, TIM-3.

In one aspect, a population of cells is contacted with a detection reagent that specifically binds one or more of the following cell surface markers: CD28, CD27, CTLA-4, CTLA-4 total, CD57, LAG3, PD-1, ICOS, BTLA, and TIM-3. In one aspect, the cell is captured on anti-CD4 bead, anti-CD8 bead, or a combination thereof. In one aspect, the labeled targeting reagent is released, eluted from the bead and captured onto an assay surface on which a targeting reagent complement is immobilized, and detected.

In one aspect, the target analyte is a macromolecular biological complex, including, but not limited to: a sub-cellular component, for example mitochondria, ribosomes, endoplasmic reticulum, Golgi apparatus, cytoskeleton, vesicle, nuclei, and the like; a lipid vesicle, for example, an exosome or low density lipoprotein (LDL) complex; nanoparticle; or a polymer composites.

### D. Detection Reagent

In one aspect, the method or kit includes a detection reagent that is capable of specifically binding to a target analyte in a sample. In one aspect, the detection reagent includes an analyte binding portion, a label and a targeting reagent. In one aspect, two or more detection reagents are used in a multiplex assay. In one aspect, a plurality of detection reagents are used in a multiplex assay.

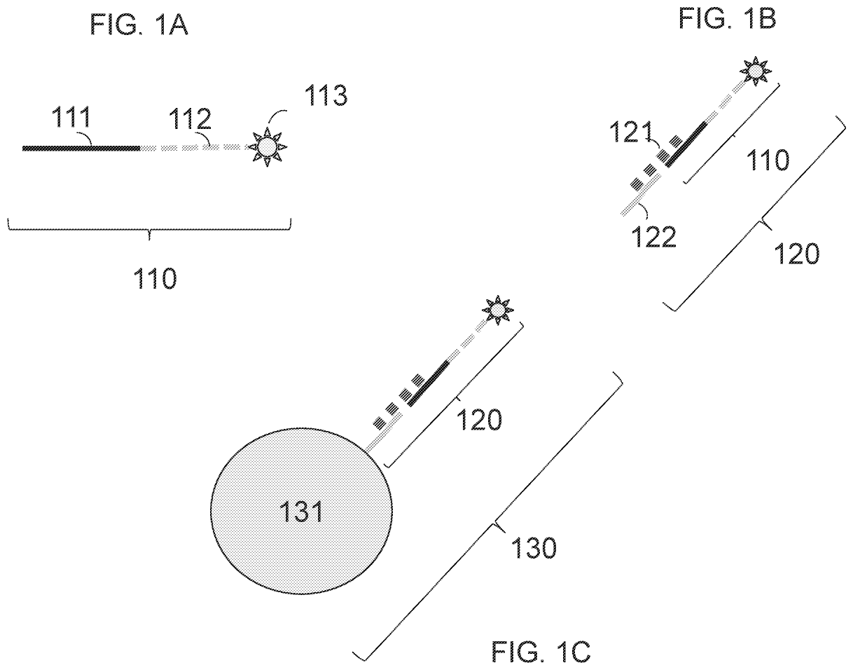

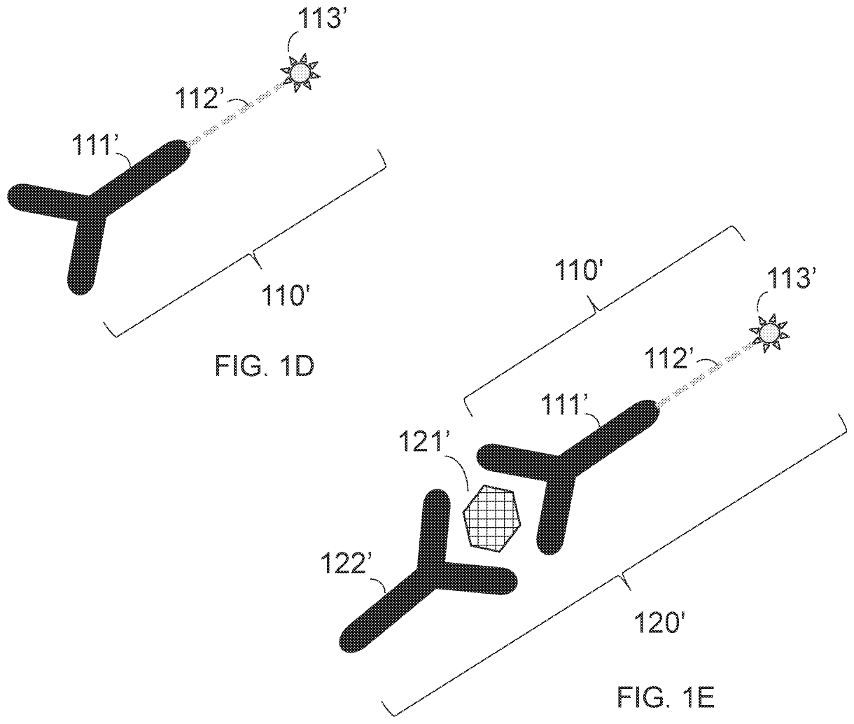

FIG. 1A provides schematic of a detection reagent 110 that includes an analyte binding portion 111, a targeting reagent 112 and a detectable label 113. In one aspect, the analyte binding portion 111 includes an oligonucleotide with a nucleic acid sequence that is complementary to a nucleic acid sequence of a target analyte. In one aspect, the targeting reagent 112 includes an oligonucleotide sequence that is complementary to an oligonucleotide sequence of a targeting reagent complement (not shown). In one aspect, the analyte binding portion 111 includes an oligonucleotide with a nucleic acid sequence that is complementary to a nucleic acid sequence of a target analyte and the targeting reagent 112 includes an oligonucleotide sequence that is complementary to an oligonucleotide sequence of a targeting reagent complement. In one aspect, the analyte binding portion 111 includes an oligonucleotide with a nucleic acid sequence that is complementary to a nucleic acid sequence of a target analyte; the targeting reagent 112 includes an oligonucleotide sequence that is complementary to an oligonucleotide sequence of a targeting reagent complement; and the label 113 includes an ECL label. In one aspect, the analyte binding portion 111 includes an oligonucleotide with a nucleic acid sequence that is complementary to a nucleic acid sequence of a target analyte; the targeting reagent 112 includes an oligonucleotide sequence that is complementary to an oligonucleotide sequence of a targeting reagent complement; and the label 113 includes a fluorescent label. In one aspect, the analyte binding portion 111 includes an oligonucleotide with a nucleic acid sequence that is complementary to a nucleic acid sequence of a target analyte; the targeting reagent 112 includes an oligonucleotide sequence that is complementary to an oligonucleotide sequence of a targeting reagent complement; and the label 113 includes a primer for rolling circle amplification. In one aspect, the analyte binding portion 111 includes an oligonucleotide with a nucleic acid sequence that is complementary to a nucleic acid sequence of a target analyte; the targeting reagent 112 includes an oligonucleotide sequence that is complementary to an oligonucleotide sequence of a targeting reagent complement; and the label 113 includes a hapten such as biotin, avidin, or streptavidin.

In one aspect, the detection reagent includes an analyte binding portion that is a protein. In one aspect, the analyte binding portion is an antibody or an antigen binding antibody fragment. In one aspect, the analyte binding portion is a receptor. In one aspect, the analyte binding portion is a receptor ligand.

FIG. 1D provides schematic of an alternate detection reagent 110′ that includes an analyte binding portion 111′, a targeting reagent 112′ and a detectable label 113′, wherein the analyte binding portion 111′ includes an antibody or an antigen binding antibody fragment. In one aspect, the analyte binding portion 111′ includes an antibody that specifically binds to a target analyte. In one aspect, the targeting reagent 112′ includes an oligonucleotide sequence that is complementary to an oligonucleotide sequence of a targeting reagent complement. In one aspect, the analyte binding portion 111′ includes an antibody that specifically binds to a target analyte and the targeting reagent 112′ includes an oligonucleotide sequence that is complementary to an oligonucleotide sequence of a targeting reagent complement. In one aspect, the analyte binding portion 111′ includes an antibody that specifically binds to a target analyte; the targeting reagent 112′ includes an oligonucleotide sequence that is complementary to an oligonucleotide sequence of a targeting reagent complement; and the label 113′ includes an ECL label. In one aspect, the analyte binding portion 111′ includes an antibody that specifically binds to a target analyte; the targeting reagent 112′ includes an oligonucleotide sequence that is complementary to an oligonucleotide sequence of a targeting reagent complement; and the label 113′ includes a fluorescent label. In one aspect, the analyte binding portion 111′ includes an antibody that specifically binds to a target analyte; the targeting reagent 112′ includes an oligonucleotide sequence that is complementary to an oligonucleotide sequence of a targeting reagent complement; and the label 113′ includes a primer for rolling circle amplification. In one aspect, the analyte binding portion 111′ includes an antibody that specifically binds to a target analyte; the targeting reagent 112′ includes an oligonucleotide sequence that is complementary to an oligonucleotide sequence of a targeting reagent complement; and the label 113′ includes a hapten such as biotin, avidin, or streptavidin.

In one aspect, the detection reagent includes a digestible linker. In one aspect, the targeting reagent is conjugated to the analyte binding portion of the detection reagent through a digestible linker. In one aspect, the targeting reagent is conjugated to the analyte binding portion of the detection reagent through a thiol group (—SH). In one aspect, the targeting reagent is conjugated to the analyte binding portion through a binding pair, for example, biotin and avidin or streptavidin.

1. Analyte Binding Portion

In one aspect, the analyte binding portion of the detection reagent is capable of specifically binding to a target analyte. In one aspect, the analyte binding portion of the detection reagent is a first member of a binding pair, wherein the target analyte is a second member of the binding pair. In one aspect, the analyte binding portion binds to a unique target analyte in the sample and does not substantially cross-react with other components or analytes in the sample. The analyte binding portion can be any naturally occurring or synthetic biological or chemical molecule which can specifically bind to a target analyte. In one aspect, the observed cross-reactivity of a particular target analyte for an analyte binding portion that does not “specifically bind” to the target analyte is less than 1%, less than 0.5%, or less than 0.1% of the binding of the particular target analyte to the corresponding analyte binding portion that “specifically binds” to the target analyte.

In one aspect, binding between the analyte binding portion and the target analyte is covalent. In one aspect, binding between the analyte binding portion and the target analyte is non-covalent. In one aspect, the analyte binding portion and the target analyte are members of a binding pair, including, but not limited to, (in either order) oligonucleotide-complementary oligonucleotide, receptor-ligand, antibody-antigen, antibody-hapten, antibody-epitope, antibody-mimitope, aptamer-target molecule, natural or synthetic receptor-ligand pairs, amines and carbonyl compounds (for example, that can bind through the formation of a Schiff s base), and intercalater-target molecule pairs. In one aspect, the analyte binding portion is selected from a peptide, a protein, an antibody or an antigen binding antibody fragment, an antibody mimetic, an affibody, a ribo- or deoxyribo-nucleic acid sequence, an aptamer, a lipid, a polysaccharide, a lectin, or a chimeric molecule formed of multiples of the same or different analyte binding molecule.

In one aspect, the analyte binding portion of the detection reagent includes a nucleotide sequence that is complementary to a nucleotide sequence of a target analyte. In one aspect, the analyte binding portion of the detection reagent includes a nucleotide sequence that is complementary to a nucleotide sequence of a target analyte. In one aspect, the nucleotide sequence of the analyte binding portion is about 5 to about 50 nucleotides in length. In one aspect, the analyte binding portion is at least about 90%, 95%, 96%, 97%, 98%, 99% or 100% identical to a nucleotide sequence of a target analyte. In one aspect, the analyte binding portion of the detection reagent includes a nucleotide sequence with a length from about 5, about 10, about 15, about 20 or about 25 nucleotides and up to about 30, about 40 or about 50 nucleotides, or about 5, about 10, about 15, about 20, about 25, about 30, about 35, about 40, about 45, or about 50 nucleotides in length. In one aspect, the nucleotide sequences of the analyte binding portion and the analyte hybridize along their entire length. In one aspect, the nucleotide sequences of the analyte binding portion and the analyte do not hybridize along their entire length. In one aspect, one of the analyte binding portion or the analyte have a shorter nucleotide sequence than the other member of the binding pair and can hybridize to a portion of the other member of the binding pair.

In one aspect, the analyte binding portion includes a polypeptide. In one aspect, the analyte binding portion includes a protein. In one aspect, the analyte binding portion includes a recombinantly produced protein. In one aspect, the analyte binding portion includes a naturally occurring protein. In one aspect, the analyte binding portion is an antibody or an antibody fragment. As used herein, an antibody fragment or antigen binding antibody fragment refers to a portion of an antibody that specifically binds to a target analyte and includes, but is not limited to Fab, Fab′, F(ab′)2, Fv fragment, single-chain Fv (scFv), scFv-Igs, or other fragments or portions of an antibody that can specifically bind to a target analyte. Additional examples of an analyte binding portion include, but are not limited to, diabody (Dab), synbody, nanobodies, BiTEs, SMIPs, DARPins, DNLs, Duocalins, adnectins, fynomers, Kunitz Domains Albu-dabs, DARTs, DVD-IG, Covx-bodies, peptibodies, scFv-Igs, SVD-Igs, dAb-Igs, Knob-in-Holes, triomAbs, or combinations thereof. In one aspect, the analyte binding portion includes a monoclonal or polyclonal antibody, or an antigen binding antibody fragment.

In one aspect, the analyte binding portion includes a receptor or a binding portion thereof that specifically binds to a target analyte that is a ligand of the receptor. In one aspect, the analyte binding portion includes a ligand that specifically binds to a target analyte that is a receptor for the ligand.

2. Targeting Reagent

In one aspect, the detection reagent includes a targeting reagent that is configured to specifically bind to a targeting reagent complement. In one aspect, the detection reagent includes a targeting reagent that is configured to specifically bind to a targeting reagent complement that is immobilized on an assay surface. In one aspect, the targeting reagent and targeting reagent complement are members of a binding pair, including, but not limited to, binding pairs such as oligonucleotide-complementary oligonucleotide, a receptor-ligand, antigen-antibody, hapten-antibody, epitope-antibody, mimetope-antibody, aptamer-target molecule pair, hybridization partners, or an intercalator-target molecule pair. In one aspect, the targeting reagent complement is an oligonucleotide that can be immobilized on an assay surface and is designed to hybridize to a complementary nucleotide sequence of a targeting reagent. In one aspect, the targeting reagent complement is a single stranded oligonucleotide that can selectively hybridize, for example, under stringent hybridization conditions, with a single stranded targeting reagent.

In one aspect, the targeting reagent and targeting reagent complement are selected such that the targeting reagent “specifically binds” to its corresponding targeting reagent complement. In one aspect, the targeting reagent and targeting reagent complement associated with an analyte (for example, a first analyte) is substantially non-cross-reactive with the targeting reagent and targeting reagent complement associated with another analyte in the sample (for example, a second analyte). In one aspect, a targeting reagent specifically binds to its corresponding targeting reagent complement and does not substantially cross-react with other targeting reagent complements that are immobilized on an assay surface. In one aspect, the observed cross-reactivity of a particular targeting reagent for targeting reagent complement that is not its corresponding targeting reagent complement is less than 1%, less than 0.5%, or less than 0.1% of the binding of the particular targeting reagent to its corresponding targeting reagent complement.

In one aspect, the targeting reagent includes an oligonucleotide sequence and its corresponding targeting reagent complement includes a complementary oligonucleotide sequence. In one aspect, the targeting reagent includes a sequence that specifically binds to an oligonucleotide sequence of a corresponding targeting reagent complement. In one aspect, the targeting reagent includes a single stranded oligonucleotide with a nucleotide sequence that is complementary to at least a portion of the nucleotide sequence of a single stranded targeting reagent complement. In one aspect, the targeting reagent is recombinantly produced. In one aspect, the nucleotide sequence of the targeting reagent and/or the targeting reagent complement are not naturally occurring sequences. In one aspect, the targeting reagent and/or targeting reagent complement include deoxyribonucleic acids (DNA), ribonucleic acids (RNA), or structural analogs that include non-naturally occurring chemical structures that can also participate in hybridization reactions.

In one aspect, the targeting reagent and/or the targeting reagent complement include an oligonucleotide with a length of at least about 5, about 10, about 15, about 20 or about 25 bases and up to about 25, about 30, about 35, about 40, about 45, about 50 or about 100 bases. In one embodiment, the targeting reagent and/or targeting reagent complement include an oligonucleotide with a length about 10 to about 50 bases, about 20 to about 40 bases, about 10 to about 25 bases, or about 15 to about 25 bases. The targeting reagent and the targeting reagent complement oligonucleotide sequences need not be identical in length and in certain embodiments it may be beneficial to provide a targeting reagent oligonucleotide sequence that is longer than its corresponding targeting reagent complement, for example, by up to about 5, about 10, about 15, about 20 or about 25 bases.

In one aspect, the nucleotide sequences of the targeting reagent and targeting reagent complement are 100% complementary. In one aspect, the nucleotide sequences of the targeting reagent and the targeting reagent complement have at least about 90%, 95%, 96%, 97%, 98% or 99% sequence complementarity along at least a portion of consecutive nucleotides. In one aspect, the nucleotide sequences of the targeting reagent and targeting reagent complement hybridize along their entire length. In one aspect, the nucleotide sequences of the targeting reagent and targeting reagent complement do not hybridize along their entire length. In one aspect, one of the targeting reagent or targeting reagent complement has a shorter nucleotide sequence than the other member of the binding pair and can hybridize to a portion of the other member of the binding pair.

In one aspect, the targeting reagent is attached to the 5′-end of the analyte binding portion of the detection reagent. In another aspect, the targeting reagent is attached to the 3′-end of the analyte binding portion of the detection reagent. In one aspect, the targeting reagent has a nucleotide sequence that is not complementary to and does not hybridize with a nucleotide sequence of a target analyte.

In one aspect, the analyte binding portion and the targeting reagent portion of the detection reagent are present on one nucleic acid strand. In another aspect, the analyte binding portion and the targeting reagent portion are present on different nucleic acid strands. In one aspect, the detection reagent includes a first strand that includes the analyte binding portion and a first bridging sequence and a second strand that includes the targeting reagent portion and a second bridging sequence complementary to the first bridging sequence, wherein the first and second strands are hybridized or can hybridize through the first and second bridging sequences.

3. Detectable Label

In one aspect, detection reagent includes a detectable label. In one aspect, the label is attached directly to the detection reagent. In another aspect, the label is attached to the detection reagent through a linker. In one aspect, the label includes a radioactive, fluorescent, chemiluminescent, electrochemiluminescent, light absorbing, light scattering, colorimetric, electrochemical, magnetic or enzymatic label.

In one aspect, the label is attached to the 5′ terminal nucleotide of the targeting reagent. In another aspect, the label is attached to the 3′ terminal nucleotide of the targeting reagent. In one aspect, the label is attached along the length of the targeting reagent.

In one aspect, the label includes an electrochemiluminescent (ECL) label. ECL labels, include, but are not limited to: i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds.

Species that participate with the ECL label in the ECL process are referred to herein as ECL coreactants. Commonly used coreactants include tertiary amines (e.g., see U.S. Pat. No. 5,846,485 and U.S. Provisional Application No. 62/787,892, filed on Jan. 3, 2019), oxalate, and persulfate for ECL from RuBpy and hydrogen peroxide for ECL from luminol (see, e.g., U.S. Pat. No. 5,240,863).

In one aspect, the label includes an organometallic complex that includes a transition metal. In one aspect, the transition metal includes ruthenium. In one aspect, the label is a MSD SULFO-TAG™ label (available from Meso Scale Diagnostics LLC, Rockville, MD).

In one aspect, the label includes a fluorescent label, including, but not limited to, fluorescein isothiocyanate (FITC), phycoerythrin (PE), allophycocyanin (APC), coriphosphine-O (CPO) or tandem labels, PE-cyanin-5 or -7 (PC5 or PC7)), PE-Texas Red (ECD), PE-cyanin-5.5, rhodamine, PerCP, Alexa labels and combinations thereof. In one aspect, a target analyte is labeled with two or more different fluorescent labels and is distinguishable from other labeled analyte by the ratio between the two or more fluorescent labels.

In one aspect, the label includes a hapten, such as biotin, avidin or streptavidin. In one aspect, the label includes biotin and avidin or streptavidin is used as a labeling reagent. In one aspect, avidin or streptavidin is labeled with a detectable label. In one aspect, avidin or streptavidin is labeled with an ECL label. In one aspect, avidin or streptavidin is labeled with a MSD SULFO-TAG™ label.

In one aspect, the label includes a primer for rolling circle amplification (RCA primer). In one aspect, the RCA primer includes an oligonucleotide with a length from about 5 to about 50 bases. In one aspect, the RCA primer is a single stranded oligonucleotide with a nucleic acid sequence that is complementary to a nucleic acid sequence of a circular RCA template. In one aspect, the RCA primer can hybridize to a complementary sequence in a circular RCA template. In one aspect, the primer is extended to form an RCA product. In one aspect, the RCA primer is extended by a polymerase. In one aspect, the RCA primer has a free 3′ end and can function as a primer for a DNA polymerase.

In one aspect, the label includes both an ECL label and an RCA primer, such that a single reagent can be used to run un-amplified assay formats and/or RCA formats.

In one aspect, the label includes both a hapten, such as a biotin moiety and an RCA primer such that the target analyte can be detected either via labeled streptavidin or via an RCA based signal amplification module.

In one aspect, the label includes an enzyme. In one aspect, the label includes an enzyme that catalyzes conversion of a substrate into a detectable product. Examples of enzymes commonly used as labels include horseradish peroxidase (HRP), alkaline phosphatase (AP), glucose oxidase and β-galactosidase. In one aspect, the label is a self-labeling enzyme.

4. Digestible Linker

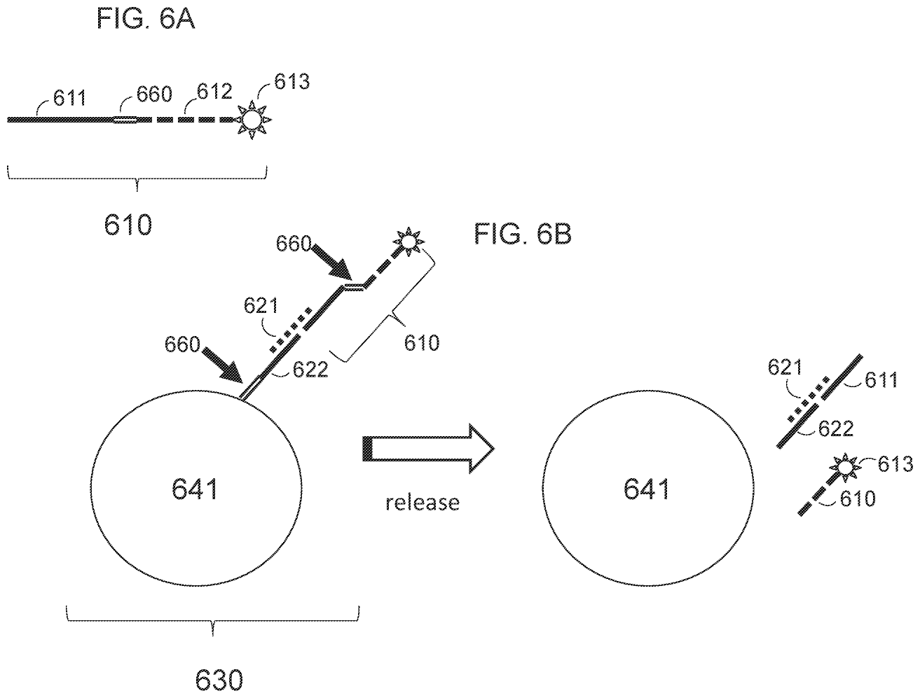

In one aspect, the detection reagent includes a digestible linker. FIG. 6A provides schematic of one embodiment of a detection reagent 610 that includes an analyte binding portion 611 that binds to an analyte 621, a targeting reagent 612, a detectable label 613 and a digestible linker 660. In one aspect, the digestible linker 660 is selected such that the targeting reagent 612 and label 613 remain linked after digestion. In one aspect, the digestible linker 660 is located between the analyte binding portion 611 and the targeting reagent 612 of the detection reagent 610.

In one aspect, digestible linker includes a nucleic acid sequence. In one aspect, the digestible linker includes a restriction recognition site. In one aspect, the digestible linker includes a peptide sequence. In one aspect, the digestible linker includes a disulfide bond.

In one aspect, the digestible linker includes a peptide sequence or a nucleotide sequence that is not present elsewhere in the detection reagent. In one aspect, the digestible linker includes a peptide sequence that is not present elsewhere in the detection reagent. In one aspect, the digestible linker includes a peptide sequence and the targeting reagent, the label and the analyte binding portion are oligonucleotides. In one aspect, the digestible linker includes a nucleotide sequence that is not present elsewhere in the detection reagent. In one aspect, the digestible linker includes an oligonucleotide that includes a restriction site not present elsewhere in the detection reagent.

5. Carrier Protein

In one embodiment, the detection reagent includes one label. In one embodiment, the detection reagent includes more than one label, for example, from about 2 to about 10 labels, or about 2, about 3, about 4, about 5, about 6, about 7, about 8, about 9 or about 10 labels. In one aspect, the detection reagent includes one ECL label. In one aspect, the detection reagent includes more than one ECL label, for example, from about 2 to about 10 ECL labels, or about 2, about 3, about 4, about 5, about 6, about 7, about 8, about 9 or about 10 ECL labels.

In one aspect, the detection reagent is conjugated to a carrier protein. In one aspect, two or more labeled targeting reagents are conjugated to the carrier protein. In one aspect, multiple copies of a labeled targeting reagent are conjugated to the carrier protein. In one aspect, the detection reagent includes a carrier protein that includes at least about 5, about 6, about 7, about 8, about 9, about 10 and up to about 15, about 20, about 25 or about 30 copies of a labeled targeting reagent. In one aspect, the detection reagent includes a carrier protein that includes at least about 5, about 6, about 7, about 8, about 9, about 10 and up to about 15, about 20, about 25 or about 30 copies of an ECL labeled targeting reagent.

In one aspect, the carrier protein is digested with a digestion reagent to release labeled targeting reagent to amplify the signal for detection. In one aspect, digestion of the carrier protein releases multiple labeled detection reagents to amplify the signal for detection. In one aspect, the labeled detection reagent released from the carrier protein amplify the signal for detection by at least about 5×, about 10×, about 20×, about 30×, about 40× and up to about 50×.

In one aspect, the carrier protein includes bovine serum albumin (BSA).

In one aspect, the carrier protein is digested with a digestion reagent after the target analyte is bound to the detection reagent. In one aspect, the carrier protein is digested after the sandwich components are released from the particulate support surface. In one aspect, the carrier protein is digested before the detection step.

In one aspect, the digestion reagent includes an enzyme. In one aspect, the digestion reagent includes a protease. In one aspect, the labeled targeting reagent is conjugated to the carrier protein through a disulfide bond. In one aspect, the digestion reagent includes a reducing agent. In one aspect, the reducing agent is selected from β-mercaptoethanol (BME) and dithiothritol (DTT).

In one aspect, the targeting reagent and label of the detection reagent are not digested by the digestion reagent. In one aspect, the targeting reagent is an oligonucleotide that is not digested by the digestion reagent. In one aspect, the label is an oligonucleotide that is not digested by the digestion reagent.

### E. Capture Molecule

As used herein, “capture molecule” refers to a molecule that specifically binds to a target analyte. In one aspect, the capture molecule is or can be immobilized to a support surface. In one aspect, the capture molecule is or can be immobilized to a particulate support surface. In one aspect, the capture molecule binds to a target analyte in a sample. In one aspect, the capture molecule specifically binds to a particular target analyte in the sample and does not substantially cross-react with other components or analytes in the sample. In one aspect, the observed cross-reactivity of a particular target analyte for a capture molecule that does not “specifically bind” to the target analyte is less than 1%, less than 0.5%, or less than 0.1% of the binding of the particular target analyte to its corresponding capture molecule that “specifically binds” to the target analyte.

In one aspect, the capture molecule and the target analyte are members of a binding pair, including, but not limited to, (in either order) oligonucleotide-complementary oligonucleotide, receptor-ligand, antibody-antigen, antibody-hapten, antibody-epitope, antibody-mimitope, aptamer-target molecule, natural or synthetic receptor-ligand pairs, amines and carbonyl compounds (for example, that can bind through the formation of a Schiff s base), and intercalater-target molecule pairs.

In one aspect, the binding site for the analyte binding portion of the detection reagent on the target analyte is different than the binding site for the capture molecule on the target analyte. In one aspect, the binding portion of the detection reagent includes an oligonucleotide with a nucleic acid sequence that is complementary to a nucleic acid sequence of the target analyte. In one aspect, the capture molecule includes an oligonucleotide with a nucleic acid sequence that is complementary to a nucleic acid sequence of the target analyte. In one aspect, the binding portion of the detection reagent includes an oligonucleotide with a nucleic acid sequence that is complementary to a first nucleic acid sequence of the target analyte and the capture molecule includes a nucleic acid sequence that is complementary to a second nucleic acid sequence of the target analyte. In one aspect, the first and second nucleic acid sequences of the target analyte to which the binding portion and the capture molecule are complementary are different. In one aspect, the first and second nucleic acid sequences of the target analyte to which the binding portion and the capture molecule are complementary do not overlap.

In one aspect, the binding portion of the detection reagent includes an antibody or an antigen binding portion thereof that specifically binds to a target analyte. In one aspect, the binding portion of the detection reagent includes an antibody or an antigen binding portion thereof that specifically binds to an epitope on the target analyte. In one aspect, the capture molecule includes an antibody or an antigen binding portion thereof that specifically binds to the target analyte. In one aspect, the capture molecule includes an antibody or an antigen binding portion thereof that specifically binds to an epitope on the target analyte. In one aspect, the binding portion of the detection reagent includes an antibody or an antigen binding portion thereof that specifically binds to a first epitope on the target analyte and the capture molecule includes an antibody or an antigen binding portion thereof that specifically binds to a second epitope on the target analyte. In one aspect, the first and second epitopes of the target analyte to which the binding portions of the detection reagent and the capture molecule specifically bind are different. In one aspect, the first and second epitopes of the target analyte to which the binding portions of the detection reagent and the capture molecule specifically bind do not overlap.

The number of capture molecules associated with a particulate support surface can vary. In one aspect, a single particulate support surface can include two or more different capture molecules that specifically bind to different target analytes. In one aspect, a single particulate support surface can include two or more copies of the same capture molecule. In one aspect, a single particulate support surface can include up to about 100, up to about 1,000, up to about 10,000, or more than 10,000 capture molecules.

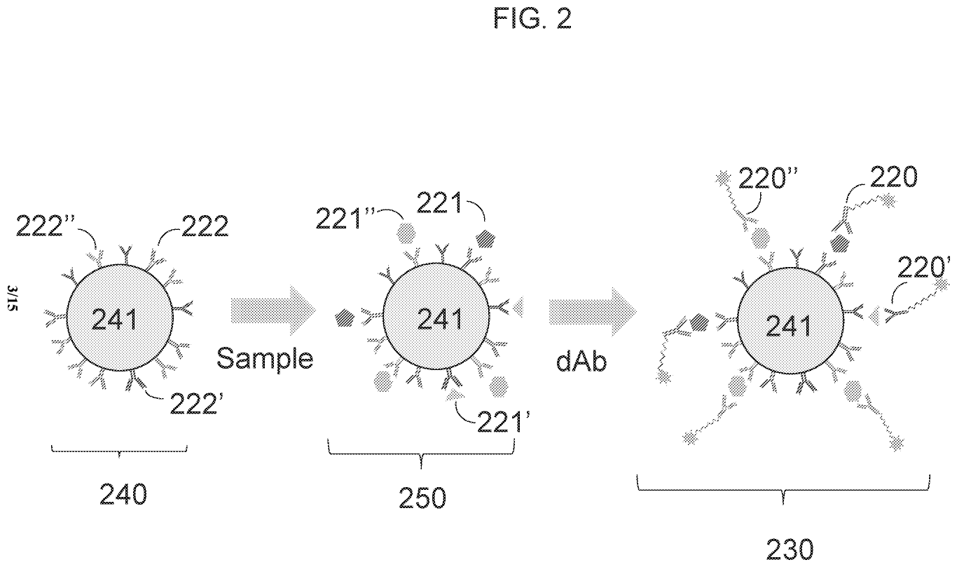

As shown schematically in FIG. 2, a plurality of different capture molecules 222, 222′, and 222″ can be immobilized on a particulate support surface 241 to form a coated particle 240. In one aspect, multiple copies of two or more different capture molecules 222, 222′, and 222″ that specifically bind to different target analytes 221, 221′, and 221″, respectively, are present on a single particulate support surface 241. In one aspect, a single particulate support surface 241 can include up to about 100 copies, up to about 1,000 copies, up to about 10,000 copies, or more than 10,000 copies of two or more different capture molecules.

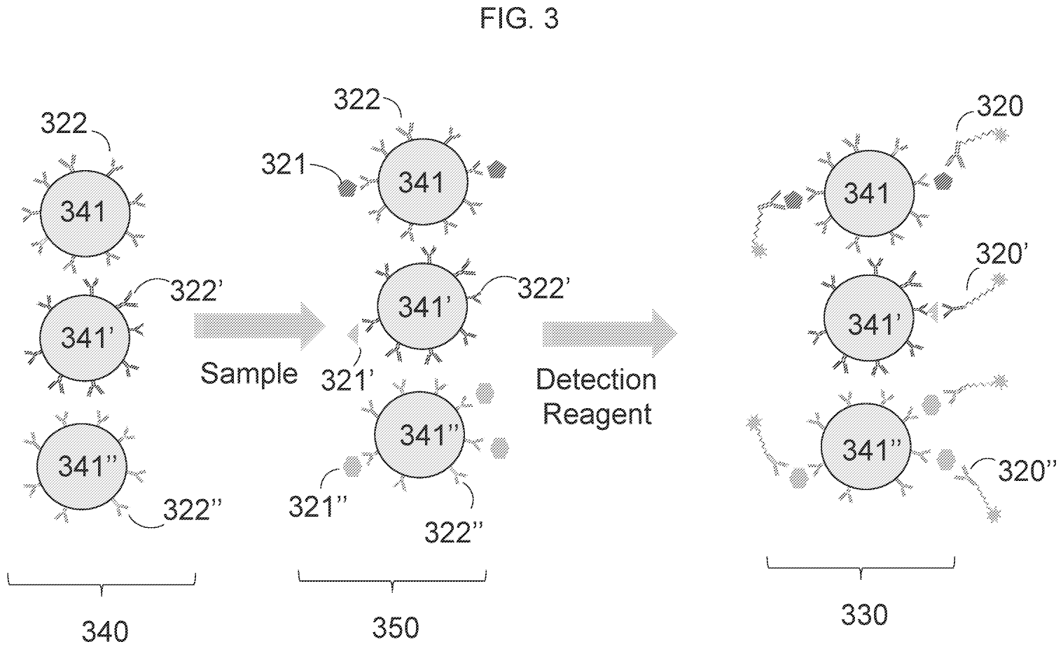

In one aspect, one or more, or each particulate support surface includes only one type of capture molecule that specifically binds one particular analyte. As shown schematically in FIG. 3, multiple copies of one type of capture molecule 322, 322′, or 322″ are immobilized on a particulate support surface 341, 341′, or 341″, respectively. In another aspect, a single particulate support surface includes multiple copies of one type of capture molecule that specifically binds one particular analyte. For example, a single particulate support surface can include up to about 100 copies, up to about 1,000 copies, up to about 10,000 copies, or more than 10,000 copies of one type of capture molecule.

In one aspect, the capture molecule or sandwich structure is covalently immobilized on the particulate support surface. In one aspect, the capture molecule or sandwich structure includes an immobilization reagent. In one aspect, the capture molecule includes an immobilization reagent. In one aspect, the capture molecule is immobilized to the support surface through an immobilization reagent. In one aspect, the immobilization reagent includes a reactive functional group. In one aspect, the immobilization reagent includes a member of a binding pair and the particulate support surface is coated with the other member of the binding pair. In one aspect, the immobilization reagent and the particulate support surface include members of binding pairs including, but not limited to, (a) a thiol group and a maleimide or iodoacetamide; (b) an aldehyde and a hydrazide; (c) an alkyne and an azide; (d) biotin and streptavidin or avidin; (e) a peptide and an anti-peptide antibody, (f) an amine group and NHS ester, (g) carboxyl group and carboxyl reactive crosslinkers, or (h) other commercially available cross-linker reagents. In one aspect, the particulate support surface includes protein A or protein G, which readily bind the Fc regions of many types of antibodies and the immobilization reagent includes an Fc region. In one aspect, the immobilization reagent includes biotin and the particulate support surface includes a commercially available streptavidin coated microparticles.

In one aspect, a capture molecule is directly immobilized to the support surface. In one aspect, the capture molecule or sandwich structure is immobilized to the support surface through a linker. In one aspect, the capture molecule or sandwich structure is immobilized to the support surface through a digestible linker. In one aspect, a capture molecule is immobilized to the support surface through a reactive functional group. In one aspect, a capture molecule is directly immobilized to the support surface through a reactive functional group. In one aspect, the capture molecule or sandwich structure is immobilized to the support surface through a reactive functional group that is attached to the capture molecule or sandwich structure through a linker. In one aspect, the capture molecule is immobilized to the support surface through a digestible linker.

In one aspect, the capture molecule or sandwich structure is non-covalently immobilized to the particulate support surface. In one aspect, the capture molecule or sandwich structure is adsorbed to the particulate support surface via electrostatic interactions, for example, between a negatively charged phosphate group on the capture molecule and a positive charge on the particulate support surface.

In one aspect, the capture molecule or sandwich structure is immobilized to a particulate support surface that has been pretreated with a protein such as Bovine Serum Albumin (BSA). In another aspect, the capture molecule or sandwich structure is immobilized to the particulate support surface through a cross-linking agent. Suitable homo-bifunctional and hetero-bifunctional cross-linking agents for connecting proteins and nucleic acids to each other or to other materials are known in the art, see for example, the Thermo Scientific Crosslinking Technical Handbook, published by Thermo Fisher Scientific, 2012). In one aspect, the cross-linking agent is a hetero-bifunctional cross-linking agent that includes an amine reactive moiety (such as an N-hydroxysuccinimide or N-hydroxysulfosuccinimide ester) and a thiol-reactive moiety such as a maleimide, an iodosuccinimide or an activated disulfide (such as a pyridyldisulfide); such hetero-bifunctional cross-functional cross-linking agents include, for example, sulfosuccinimidyl-4-(N-maleimidomethyl)cyclohexane-1-carboxylate (Sulfo-SMCC). In one aspect, the amine reactive moiety (for example, the N-hydroxysuccinimide (NETS) moiety of SMCC) is reacted with a protein to introduce thiol-reactive moieties (for example, the maleimide moiety of SMCC) into the protein.

### F. Method

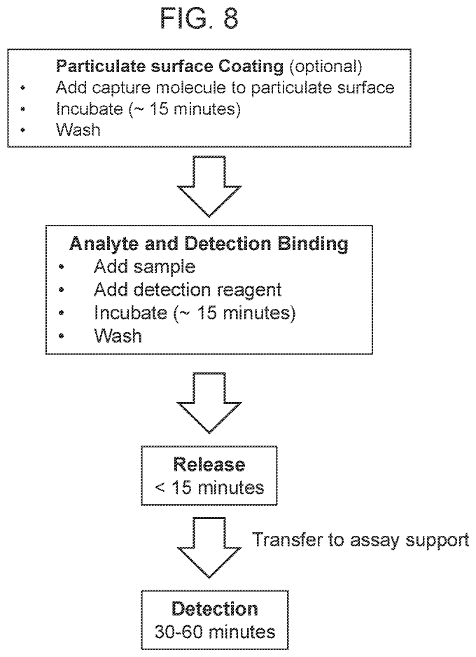

In one aspect, a method of detecting or quantifying one or more target analytes in one or more samples is provided. In one aspect, a method of detecting or quantifying a plurality of target analytes in one or more samples is provided. In one aspect, a method of conducting a multiplexed binding assay for a plurality of target analytes in a sample is provided. In one aspect, the method includes an analyte binding step and a detection step. In one aspect, the analyte binding step and detection step are performed separately, for example, using different support surfaces and/or at different times. In one aspect, shown in FIG. 8, the method includes a combined analyte and detection reagent binding step, followed by a release step. In one aspect, the method includes an optional bead coating step.

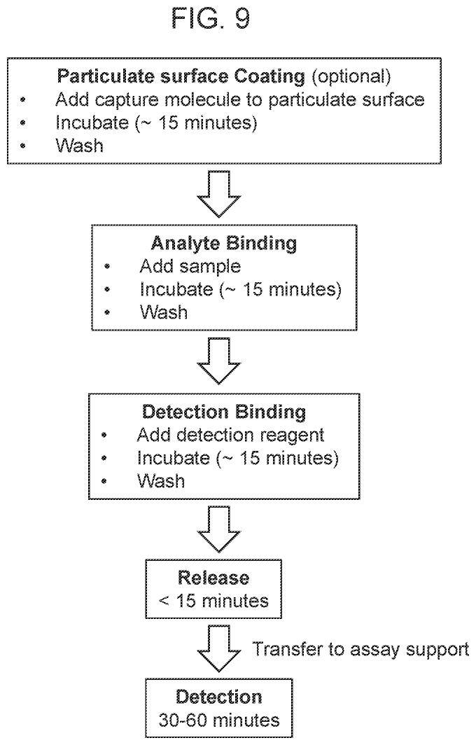

FIG. 9 provides an overview of an alternate method disclosed herein that includes an analyte binding step, followed by a detection reagent binding step and then a release step. In one aspect, the bead coating step is optional, or can be prepared in advance, and includes combining a capture molecule and a particulate support surface, incubating the mixture to allow the capture molecules to be immobilized on the particulate support surface to form coated particles and washing the coating particles to remove unbound capture molecule. In one aspect, a coated particle is formed by immobilizing one or more capture molecules on a particulate support surface.

In one aspect, the analyte binding step includes a first step in which a sample is contacted with a detection reagent that includes an analyte binding portion, a targeting reagent and incubated under conditions in which the target analyte binds to the detection reagent to form a binding complex. In one aspect, unbound analyte and/or unbound detection reagent are removed in one or more wash steps. In one aspect, the washed binding complex is contacted with a release reagent to release a detectable portion of the binding complex, wherein the detectable portion includes targeting reagent and label. In one aspect, the released detectable portion is added to an assay surface on which a targeting reagent complement is immobilized.

In one aspect, the analyte binding step includes a first step in which the coated particulate support surface is combined with a sample and incubated to allow target analyte to bind to the capture molecules immobilized on the support surface. In one aspect, the target analyte specifically binds to the capture molecule immobilized on the coated particulate support surface to form an analyte coated particle. In one aspect, the analyte coated particle is washed to remove unbound analyte. In one aspect, the analyte binding step includes a second step in which the analyte coated particle is contacted with a detection reagent and incubated to allow the detection reagent to specifically bind to the bound analyte to form a sandwich complex. In one aspect, the sandwich complex includes a sandwich structure immobilized on the particulate support surface. In one aspect, the sandwich complex is washed to remove unbound detection reagent. In one aspect, the two analyte binding steps can be combined. In one aspect, the washed sandwich complex is contacted with a release reagent to release sandwich components from the particulate support surface. In one aspect, the released sandwich components are added to an assay surface on which a targeting reagent complement is immobilized. In one aspect, the particulate support surface is removed from the released sandwich components before the released sandwich components are transferred to an assay surface on which a target reagent complement is immobilized. In one aspect, the targeting reagents of the released sandwich components are allowed to bind to the target reagent complements and the presence of labeled targeting reagent immobilized on the assay surface is detected.

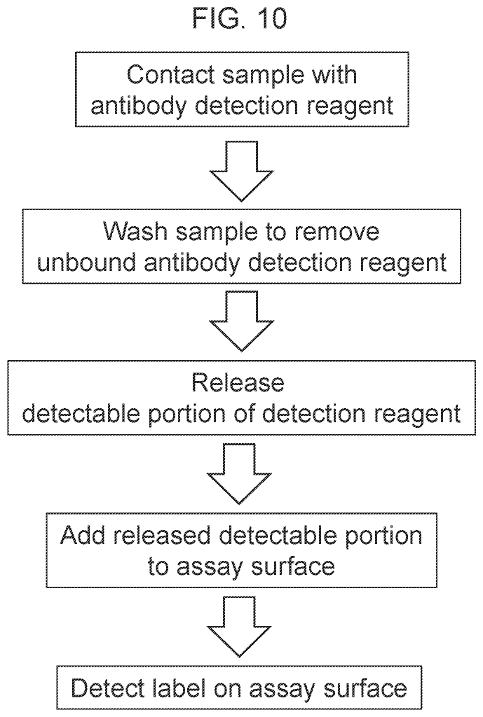

In one aspect, shown in FIG. 10, a sample is contacted with a detection reagent in which the analyte binding portion includes an antibody that specifically binds to a target analyte under conditions in which the detection reagent specifically binds to target analyte in the sample to form a binding complex. In one aspect, the sample is washed to remove unbound detection reagent. In one aspect, a detectable portion is released from the binding complex. In one aspect, the released detectable portion is added to an assay surface on which a targeting reagent complement is immobilized. In one aspect, the presence of the label of the detectable portion on the assay surface is detected.

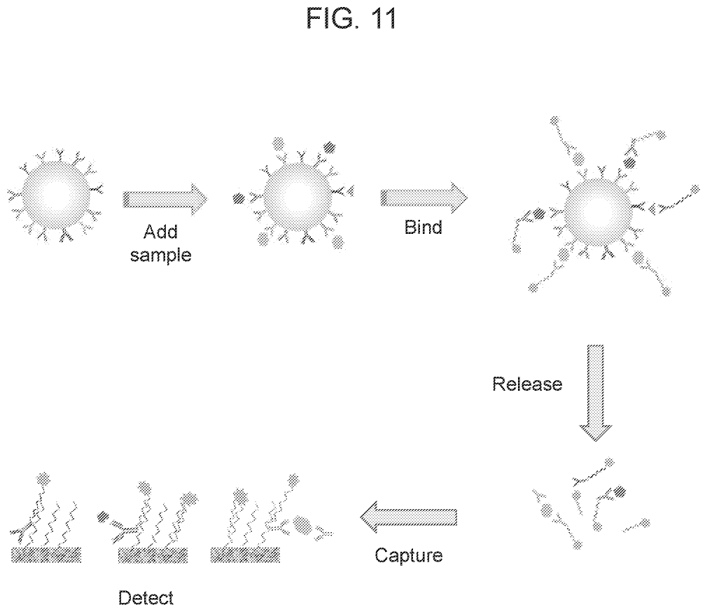

FIG. 11 provides a schematic of the method described herein. In one aspect, the analyte binding step includes a first step in which a coated particulate support surface is combined with a sample and incubated to allow target analyte to bind to the capture molecules immobilized on the support surface to form an analyte coated particle. In one aspect, the analyte binding step includes a second step in which the analyte coated particle is contacted with a detection reagent and incubated to allow the detection reagent to specifically bind to the bound analyte to form a sandwich complex. In the release step, the sandwich complex is contacted with a release reagent to release sandwich components from the particulate support surface. In the capture step, the released sandwich components are added to an assay surface on which a targeting reagent complement is immobilized. In the detection step, the targeting reagents of the released sandwich components are allowed to bind to the target reagent complements immobilized on the assay surface and the presence of labeled targeting reagent immobilized on the assay surface is detected.

In one aspect, the analyte binding portion of the detection reagent includes an oligonucleotide. In one aspect, the target analyte includes an oligonucleotide. In one aspect, the capture molecule includes an oligonucleotide. In one aspect, the targeting reagent includes an oligonucleotide. In one aspect, the analyte binding portion of the detection reagent and the targeting reagent both include an oligonucleotide. In one aspect, the nucleotide sequences of the analyte binding portion and the targeting reagent are different. In one aspect, the analyte binding portion of the detection reagent, the targeting reagent and the capture molecule all include an oligonucleotide. In one aspect, the analyte binding portion of the detection reagent, the targeting reagent, the target analyte and the capture molecule all include an oligonucleotide. In one aspect, the analyte binding portion of the detection reagent includes an oligonucleotide with a nucleotide sequence that is complementary to a nucleotide sequence of the target analyte. In one aspect, the capture molecule includes an oligonucleotide with a nucleotide sequence that is complementary to a nucleotide sequence of the target analyte. In one aspect, the analyte binding portion of the detection reagent includes a first oligonucleotide with a first nucleotide sequence that is complementary to a first nucleotide sequence of the target analyte. In one aspect, the capture molecule includes a second oligonucleotide with a second nucleotide sequence that is complementary to a second nucleotide sequence of the target analyte. In one aspect, the first and second nucleotide sequences of the analyte binding portion and the capture molecule are different. In one aspect, the first and second nucleotide sequence of the target analyte to which the analyte binding portion and the capture molecule are complementary are different. In one aspect, the first and second nucleotide sequence of the target analyte to which the analyte binding portion and the capture molecule are complementary do not overlap.

In one aspect, the analyte binding portion of the detection reagent includes a protein. In one aspect, the capture molecule includes a protein. In one aspect, the targeting reagent includes a protein. In one aspect, the targeting reagent includes an oligonucleotide. In one aspect, the analyte binding portion of the detection reagent and the capture molecule include a protein. In one aspect, the analyte binding portion of the detection reagent and the capture molecule include a protein and the targeting reagent includes an oligonucleotide. In one aspect, the analyte binding portion of the detection reagent includes a protein that specifically binds to a target analyte. In one aspect, the capture molecule includes a protein that specifically binds to a target analyte. In one aspect, the targeting reagent includes a protein that specifically binds to a targeting reagent complement. In one aspect, the analyte binding portion of the detection reagent and the capture molecule each include a protein that specifically binds to a target analyte. In one aspect, the analyte binding portion of the detection reagent and the capture molecule each include a protein that specifically binds to a target analyte and the targeting reagent includes an oligonucleotide. In one aspect, the analyte binding portion of the detection reagent includes a first protein that specifically binds to a first portion of the target analyte. In one aspect, the capture molecule includes a second protein that specifically binds to a second portion of the target analyte. In one aspect, the first and second proteins of the analyte binding portion and the capture molecule are different. In one aspect, the first and second portions of the target analyte to which the analyte binding portion and the capture molecule specifically bind are different. In one aspect, the first and second portions of the target analyte to which the analyte binding portion and the capture molecule specifically bind do not overlap.

In one aspect, the analyte binding portion of the detection reagent is a protein and the targeting reagent includes an oligonucleotide. In one aspect, the analyte binging portion of the detection reagent is an antibody or an antigen binding antibody fragment and the targeting reagent is an oligonucleotide. In one aspect, the analyte binging portion of the detection reagent is a receptor or a ligand binding receptor fragment and the targeting reagent is an oligonucleotide. In one aspect, the analyte binging portion of the detection reagent is a ligand that includes a peptide and the targeting reagent is an oligonucleotide. In one aspect, the release reagent is a protease.

In one aspect, the protein includes an antibody or an antigen binding fragment thereof. In another aspect, the protein includes a receptor or a ligand binding fragment thereof. In one aspect, the protein includes a receptor ligand.

In one aspect, the analyte binding portion of the detection reagent and the targeting reagent both include an oligonucleotide and are attached to one another through a digestible linker. In one aspect, the digestible linker includes an oligonucleotide with a restriction site that is not present in the analyte binding portion. In one aspect, the digestible linker includes an oligonucleotide with a restriction site that is not present in the targeting reagent. In one aspect, the digestible linker includes an oligonucleotide with a restriction site that is not present in the analyte binding portion or the targeting reagent. In one aspect, the digestible linker includes a restriction site that is not present in the analyte binding portion or the targeting reagent and the release reagent includes an endonuclease. In one aspect, the digestible linker includes a peptide sequence. In one aspect, the digestible linker includes a peptide sequence and the release reagent includes a protease. In one aspect, the digestible linker includes a disulfide bond. In one aspect, the digestible linker includes a disulfide bond and the release reagent includes a reducing agent.

In one aspect, the digestible linker includes an oligonucleotide with a restriction site that is not present in the analyte binding portion of the detection reagent. In one aspect, the digestible linker includes an oligonucleotide with a restriction site that is not present in the targeting reagent. In one aspect, the digestible linker includes an oligonucleotide with a restriction site that is not present in the analyte binding portion or the targeting reagent. In one aspect, the digestible linker includes a restriction site that is not present in the analyte binding portion or the targeting reagent and the release reagent includes an endonuclease. In one aspect, the digestible linker includes a peptide sequence.

In one aspect, the digestible linker includes a peptide sequence and the targeting reagent does not include a peptide sequence. In one aspect, the digestible linker includes a peptide sequence and the targeting reagent includes an oligonucleotide. In one aspect, the digestible linker includes a peptide sequence and the release reagent includes a protease.

In one aspect, the digestible linker includes a disulfide bond. In one aspect, the digestible linker includes a disulfide bond and the targeting reagent does not include a disulfide bond. In one aspect, the digestible linker includes a disulfide bond and the release reagent includes a reducing agent.

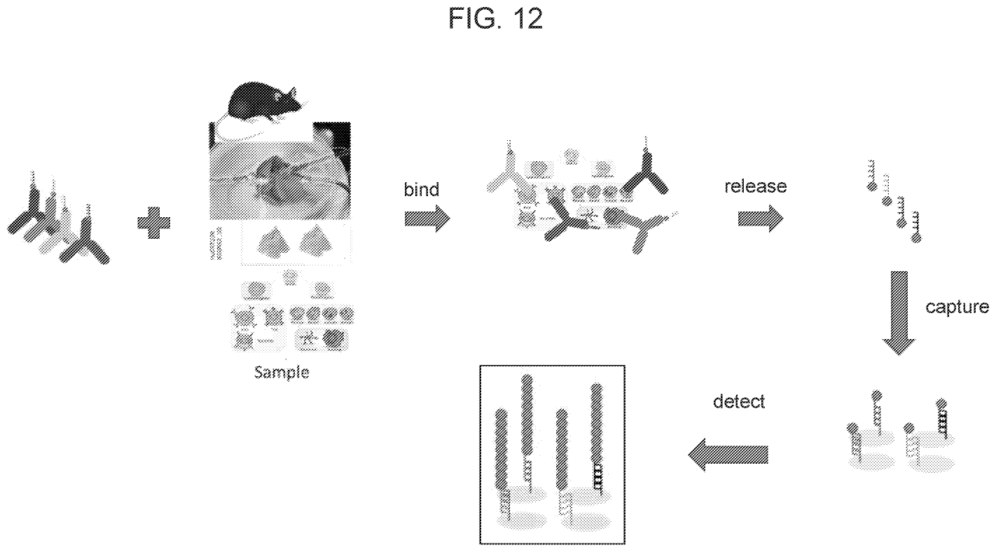

In one aspect, shown in FIG. 12, the analyte binding step includes contacting a sample, for example, a sample that includes a cell or tissue, with a detection reagent that includes an analyte binding portion that specifically binds to a target analyte, for example, a target analyte associated with the an isolated cell or a multicellular structure, for example, a tissue, organ, organelle or organism, to form a binding complex. In one aspect, the binding complex includes bound analyte and the detection reagent. In one aspect, the sample is washed to remove unbound detection reagent. In one aspect, the washed sample is contacted with a release reagent to release a detectable portion from the binding complex. In one aspect, the detectable portion includes a targeting reagent and a label. In one aspect, the released detectable portion is transferred to an assay surface on which a targeting reagent complement is immobilized under conditions in which the targeting reagent of the released detectable portion bind to the corresponding target reagent complement immobilized on the assay surface. In one aspect, the analyte binding portion includes an antibody or an antigen binding antibody fragment and the targeting reagent is an oligonucleotide. In one aspect, the analyte binding portion includes an antibody or an antigen binding antibody fragment and the targeting reagent is an oligonucleotide and the release reagent includes a protease. In one aspect, the analyte binding portion of the detection reagent is attached to a targeting reagent through a digestible linker. In one aspect, the digestible linker includes a disulfide bond. In one aspect, the digestible linker includes a restriction site and the release reagent includes an endonuclease. In one aspect, the label immobilized on the assay surface is detected.

In one aspect, the sample includes one or more cell surface markers. In one aspect, the sample includes a population of cells. In one aspect, the sample includes an isolated cell. In one aspect, the sample includes a multicellular structure, for example, a tissue, organ, organelle or organism. In one aspect, the tissues and cells in the sample are in suspension. In one aspect, the tissues and cells in the sample are not in suspension. In one aspect, the sample includes a fixed tissue sample, a live tissue sample, a perfused organ, cell culture, a whole organism, including, for example, bacteria, fungi, protozoa, algae, virus or other microorganism. In one aspect, the sample is contacted with the detection reagent in vivo, for example, by injecting the detection reagent into a live organism.

In one aspect, the label includes a fluorescent label. In one aspect, the label includes an ECL label. In one aspect, the label includes a primer for rolling circle amplification. In one aspect, the label includes a hapten such as biotin, avidin or streptavidin.

In one aspect, the analyte binding protein of the detection reagent includes a protein that specifically binds a target analyte, for example, an antibody or a receptor, and the targeting reagent includes an oligonucleotide with a nucleotide sequence that is complementary to the nucleotide sequence of a targeting reagent complement immobilized on an assay surface, wherein the oligonucleotide targeting reagent includes a haptin, such as biotin, such that avidin or streptavidin can be used as a labeling reagent. In one aspect, the avidin or streptavidin labeling reagent is labeled with an ECL label, for example, a MSD SULFO-TAG® label (Meso Scale Discovery LLC, Rockville, MD). In one aspect, the avidin or streptavidin labeling reagent is directly labeled with an ECL label, for example, a MSD SULFO-TAG® label. In one aspect, the avidin or streptavidin labeling reagent is labeled with an RCA primer. In one aspect, the avidin or streptavidin labeling reagent is labeled with a fluorescent label. In one aspect, the avidin or streptavidin labeling reagent is labeled with an enzyme.

In one aspect, the analyte binding protein of the detection reagent includes a protein that specifically binds a target analyte, for example, an antibody or a receptor, and the targeting reagent includes an oligonucleotide with a nucleotide sequence that is complementary to the nucleotide sequence of a targeting reagent complement immobilized on an assay surface. In one aspect, oligonucleotide targeting reagent includes both an ECL label and an RCA primer, such that a single reagent can be used to run un-amplified assay formats and/or RCA formats. In one aspect, the oligonucleotide targeting reagent includes both a hapten, such as biotin and an RCA primer site such that a target analyte can be detected either by labeled streptavidin or an RCA based signal amplification module.

1. Analyte Binding

In one aspect, the analyte binding step includes, in one or more steps, forming a binding complex that includes a detection reagent and bound analyte. In one aspect, the binding complex is a sandwich structure that includes capture molecule, bound analyte and detection reagent. In one aspect, the binding complex is a sandwich complex that includes a particulate support surface on which a sandwich structure is immobilized.

In one aspect, the target analyte is immobilized on a support surface or associated with an isolated cell, or a multicellular structure such as a, tissue, organ, organelle or organism and the analyte binding step includes forming a binding complex that includes a detection reagent and bound analyte, wherein the bound analyte is immobilized on the support surface or associated with the cell, tissue, organ or organism.

In one aspect, analyte binding includes, in one or more steps, forming a sandwich complex that includes a particulate support surface on which a sandwich structure is immobilized.

Particulate support surfaces can have a variety of shapes and sizes, including, but not limited to, spherical, oblong, rod-like, or irregular. In one aspect, the particulate support surface is spherical. In one aspect, the particulate support surface is a microparticle. In one aspect, the particulate support surface is a microsphere. In one aspect, the particulate support surface is a bead. In one aspect, the particulate support surface has a size in the nanometer to micrometer range, for example, at least about 100 nm, about 500 nm or about 1 μm, and up to about 1 μm, about 2 μm, about 3 μm, about 4 μm, about 5 μm or about 10 μm. In one aspect, the particulate support surface has a size from about 0.5 μm to about 5 μm, or from about 1 μm to about 4 μm.

The particulate support material can vary. In one aspect, the particulate support surface is magnetically responsive. As used herein “magnetically responsive” refers to the ability of the particulate support material to migrate, relative to the surroundings, under the influence of an external magnetic field. In one aspect, the particulate support material paramagnetic. In one aspect, the particulate support material is ferromagnetic. In one aspect, the particulate support surface includes gold. In one aspect, the particulate support material includes a polymeric matrix in which a magnetic substance is incorporated. In one aspect, the particulate support surface includes a magnetic core and a polymeric coating. In one aspect, the polymer coating includes, but is not limited to, polystyrene, or polystyrene in combination with other co-polymers such as polymethylmethacrylate (PMMA), divinylbenzene, polyvinyltoluene (PVT), styrene, butadiene, vinyltoluene, latex or silica.

In one aspect, the particulate support surface is not magnetic. In one aspect, the particulate support surface includes, but is not limited to, polymeric material, ceramic, or glass. In one aspect, the particulate support surface is a polymeric coated particle. In one aspect, the particulate support surface includes polystyrene. In one aspect, the particulate support surface includes polyethylene.

In one aspect, the particulate support surface is coated with an affinity binding ligand. In one aspect, the particulate support surface is coated with an affinity binding ligand to facilitate immobilization of a capture molecule thereon. In one aspect, the affinity binding ligand is avidin, streptavidin or biotin and the capture molecule includes an immobilization reagent that includes the other member of the biotin, avidin-streptavidin binding pair. In one aspect, the particulate support surface includes a commercially available streptavidin coated microparticles and the capture molecule includes biotin as an immobilization reagent. In one aspect, the particulate support surface includes a carboxyl or amine coating.

FIG. 1B provides schematic of a sandwich structure 120 that includes a detection reagent 110, analyte 121 and capture molecule 122. FIG. 1C provides a schematic of a sandwich complex 123 that includes a sandwich structure 120 immobilized on a particulate support surface 131. In one aspect, the analyte binding portion and targeting reagent of the detection reagent are oligonucleotides. In one aspect, the capture molecule is an oligonucleotide. In one aspect, the target analyte is an oligonucleotide.

FIG. 1E provides a schematic of an alternate sandwich structure 120′ that includes a detection reagent 110′, analyte 121′ and capture molecule 122′, in which the analyte binding portion 111′ of the detection reagent 110′ and the capture molecule 122′ are antibodies.

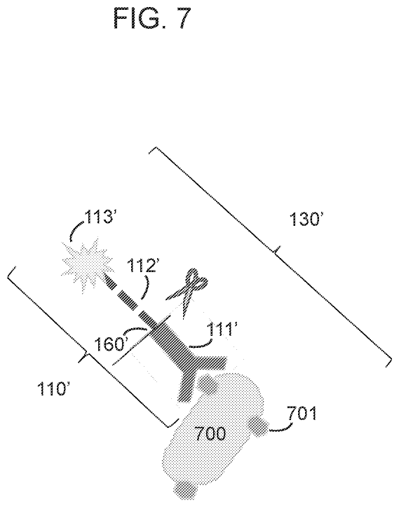

FIG. 7 provides a schematic of a binding complex 130′ that includes a detection reagent 110′ and bound analyte 701. In one aspect, the detection reagent 110′ includes an analyte binding portion 111′, a targeting reagent 112′ and a label 113′. In one aspect, the detection reagent 110′ includes a digestible linker 160′. In one aspect, the digestible linker 160′ links the analyte binding portion 111′ to the targeting reagent 112′. In one aspect, the analyte binding portion 111′ is an antibody. In one aspect, the bound analyte 701 is associated with a cell 700.

In one aspect, the analyte binding step includes, in one or more steps, combining a sample with a particulate support surface, a capture molecule and a detection reagent to form a sandwich complex that includes the particulate support surface on which a sandwich structure is immobilized.

In one aspect, the method includes a step of coating a particulate support surface with a capture molecule to form a coated particle. In one aspect, a capture molecule includes an immobilization reagent to facilitate immobilization of the capture molecule on the particulate support surface. In one aspect, the particulate support surface is coated with an affinity binding ligand, such as avidin, streptavidin or biotin. In one aspect, the immobilization reagent includes avidin, streptavidin or biotin. In one aspect, the particulate support surface is coated with streptavidin and the immobilization reagent includes biotin. In one aspect, the particulate support surface includes a carboxyl or amine coating. In one aspect, the coated particle is prepared in advance.

In one aspect, shown schematically in FIG. 2, a sandwich complex 230 is formed by combining a sample with one or more coated particles 240 that include a particulate support surface 241 on which a plurality of different capture molecules 222, 222′, 222″ are immobilized and allowing analytes 221, 221′, 221″ in the sample to bind to their corresponding capture molecules 222, 222′, 222″ to form an analyte coated particle 250 that includes the coated particle 241, capture molecules 222, 222′, 222″ and bound analyte 221, 221′, 221″. In one aspect, the coated particle 240 includes a plurality of different capture molecules 222, 222′, 222″, wherein each different capture molecule specifically binds to a different target analyte 221, 221′, 221″. In one aspect, the analyte coated particle 250 is contacted with one or more labeled detection reagents 220, 220′, 220″ to form a sandwich complex 230.

In one aspect, shown schematically in FIG. 3, a sandwich complex 330 is formed by combining a sample with a plurality of coated particles 340 that include a particulate support surface 341 on which a plurality of capture molecules 322, 322′, 322″ are immobilized and allowing analytes 321, 321′, 321″ in the sample to bind to the capture molecules 322, 322′, 322″ to form an analyte coated particle 350 that includes the coated particle 341, capture molecule 322, 322′, 322″ and bound analyte 321, 321′, 321″. In one aspect, each coated particle 340 includes only one type of capture molecule 322, 322′, 322″. In one aspect, a plurality of coated particles 340 are contacted with the sample, wherein each coated particle 340 includes only one type of capture molecule 322, 322′, 322″ and each different type of capture molecule specifically binds to a different target analyte 321, 321′, 321″. In one aspect, the analyte coated particle 350 is contacted with one or more labeled detection reagents 320, 320′, 320″ to form a sandwich complex 330.

In one aspect, the method includes a step of washing the analyte coated particle to remove unbound analyte to form a washed analyte coated particle before the analyte coated particle is contacted with the labeled detection reagent.

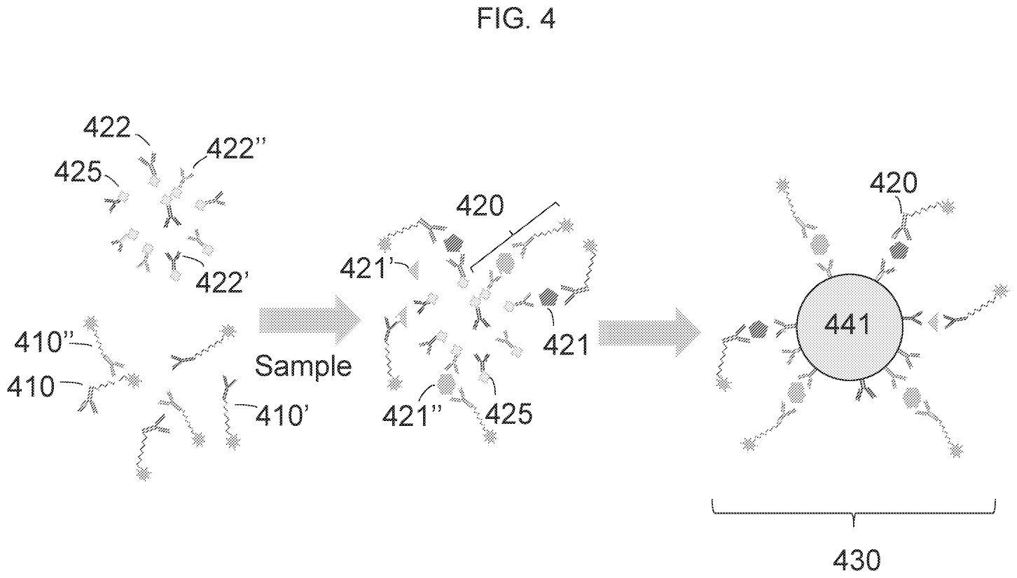

In one aspect, shown schematically in FIG. 4, a sandwich complex 430 is formed by combining a sample with a plurality of different capture molecules 422, 422′, 422″ and a plurality of different labeled detection reagents 410, 410′, 410″, and allowing analytes 421, 421′, 421″ in the sample to bind to their corresponding capture molecules 422, 422′, 422″ and detection reagents 410, 410′, 410″ to form sandwich structures 420, 420′, 420′, and then contacting one or more particulate support surfaces 441 with the sandwich structures 420 under conditions in which the sandwich structures 420 are immobilized on the particulate support surface 441 to form a sandwich complex 430.

In one aspect, the capture molecules 422, 422′, 422″ include an immobilization reagent 425. In one aspect, the resulting sandwich structures 420, 420′, 420′ are immobilized on the particulate support surface 441 through the immobilization reagent 425 to form a sandwich complex 430. In one aspect, the immobilization reagent 425 includes a reactive group. In one aspect, the immobilization reagent 425 includes a member of a binding pair and the particulate support surface 441 is coated with the other member of the binding pair. In one aspect, the immobilization reagent 425 and the particulate support surface 441 include members of binding pairs including, but not limited to, (a) a thiol group and a maleimide or iodoacetamide; (b) an aldehyde and a hydrazide; (c) an alkyne and an azide; (d) biotin and streptavidin or avidin; or (e) a peptide and an anti-peptide antibody. In one aspect, the immobilization reagent 425 includes a reactive biotin moiety and the particulate support surface 441 includes at least one streptavidin or avidin group, or vice versa. In one aspect, the particulate support surface 441 includes protein A or protein G, which readily bind the Fc regions of many types of antibodies and the immobilization reagent 425 includes an Fc region.

In one aspect, the method includes a step of washing the sandwich complex to remove unbound detection reagent, analyte, and/or capture molecule to form a washed sandwich complex before contacting the sandwich complex with a release agent to release sandwich components from the sandwich complex.

2. Release

In one aspect, the method includes a release step in which a binding complex is contacted with a release reagent to release a detectable portion from the binding complex. In one aspect, the binding complex includes a detection reagent and bound analyte. In one aspect, the detectable portion includes label and a targeting reagent that is capable of specifically binding to its corresponding targeting reagent complement. In one aspect, the method includes a release step in which a sandwich complex is contacted with a release reagent to release sandwich components from the particulate support surface. In one aspect, the release step releases labeled targeting reagent that is capable of specifically binding to its corresponding targeting reagent complement.

In one aspect, a detectable portion is released from a binding complex by contacting the binding complex with a release reagent. In one aspect, the targeting reagent includes an oligonucleotide and the analyte binding portion includes a peptide sequence. In one aspect, the targeting reagent is an oligonucleotide and the analyte binding portion includes an antibody or an antigen binding antibody fragment. In one aspect, the detectable portion is released from the binding complex by contacting the binding complex with a release reagent that denatures or digests peptide sequences but leaves oligonucleotide sequences intact. In one aspect, the detectable portion is released by contacting the binding complex with a protease.

In one aspect, a sandwich component is released from a sandwich complex by contacting the sandwich complex with a release reagent. In one aspect, the targeting reagent of the detection reagent is an oligonucleotide and the analyte binding portion and/or the capture molecule include a peptide sequence. In one aspect, the sandwich components are released from the sandwich complex by contacting the sandwich complex with a release reagent that denatures or digests peptide sequences but leaves oligonucleotide sequences intact. In one aspect, the release reagent includes a protease.

In one aspect, the targeting reagent is an oligonucleotide that is linked to the analyte binding portion through a digestible linker. In one aspect, the targeting reagent of the detection reagent is an oligonucleotide that is linked to the analyte binding portion through a digestible linker that includes a peptide sequence. In one aspect, the targeting reagent is an oligonucleotide that is linked to the analyte binding portion through an oligonucleotide that includes a restriction site. In one aspect, the restriction site of the digestible linker is not present in the targeting reagent. In one aspect, the targeting reagent is an oligonucleotide that is linked to the analyte binding portion through a digestible linker that includes a disulfide bond. In one aspect, the targeting reagent is an oligonucleotide that is linked to the analyte binding portion through a peptide that includes a disulfide bond. In one aspect, the release reagent is a reducing reagent. In one aspect, the targeting reagent is an oligonucleotide that is linked to the analyte binding portion through a peptide that includes a disulfide bond and the detectable portion is released by contacting the binding complex with a reducing reagent.

In one aspect, the protease is selected from: a serine protease, cysteine protease, threonine protease, aspartic protease, glutamic protease, and metalloprotease. In one aspect, the protease includes a serine protease. In one aspect, the protease includes proteinase K. In one aspect, the release reagent includes an endopeptidase such as pepsin or trypsin. In one aspect, the release reagent includes a reducing reagent such as ß-mercaptoethanol (BME) or dithiothritol (DTT).

In one aspect, a detectable portion is released from a binding complex by contacting the binding complex with a release reagent that includes a restriction endonuclease. In one aspect, the detection reagent includes a nucleotide sequence with a restriction site. In one aspect, the analyte binding portion of the detection reagent includes a nucleotide sequence with a restriction site. In one aspect, the detection reagent includes a nucleotide sequence with a digestible linker that includes a restriction site. In one aspect, the detection reagent includes a nucleotide sequence with a restriction site between the analyte binding portion and the targeting reagent.

In one aspect, a sandwich component is released from a sandwich complex by contacting the sandwich complex with a release reagent that includes a restriction endonuclease. In one aspect, the detection reagent includes a nucleotide sequence with a restriction site. In one aspect, the analyte binding portion of the detection reagent includes a nucleotide sequence with a restriction site. In one aspect, the detection reagent includes a nucleotide sequence with a digestible linker that includes a restriction site. In one aspect, the detection reagent includes a nucleotide sequence with a restriction site between the analyte binding portion and the targeting reagent. In one aspect, the sandwich complex includes a nucleotide sequence with a restriction site. In one aspect, the capture molecule includes a nucleotide sequence with a restriction site.

In one aspect, shown in FIG. 6A, the detection reagent 610 includes a label 613, a targeting reagent 612, an analyte binding portion 611, and a digestible linker 660. In one aspect, the analyte binding portion 611 of the detection reagent 610 includes a nucleotide sequence that is complementary to a nucleotide sequence of a target analyte (not shown) and the targeting reagent 612 includes a nucleotide sequence that is complementary to a nucleotide sequence of its corresponding targeting reagent complement (not shown). In one aspect, the analyte binding portion 611 and the targeting reagent 612 of the detection reagent 610 are linked with a digestible linker 660. In one aspect, the analyte binding portion 611 includes a first oligonucleotide sequence and the targeting reagent 612 includes a second oligonucleotide sequence. In one aspect, the digestible linker 660 includes a disulfide bond, a short peptide sequence or a protein. In one aspect, the digestible linker 660 includes a disulfide bond.

In one aspect, shown in FIG. 6B, the capture molecule 622 includes a digestible linker 660. In one aspect, the capture molecule 622 is immobilized onto the particulate support surface 641 through a digestible linker 660. In one aspect, the capture molecule 622 is an oligonucleotide and the digestible linker 660 includes a restriction site, a short peptide sequence, a protein or a disulfide bond. In one aspect, the digestible linker 660 includes an oligonucleotide with a restriction site. In one aspect, the digestible linker 660 includes a short peptide sequence. In one aspect, the digestible linker 660 includes a protein. In one aspect, the digestible linker 660 includes a disulfide bond.

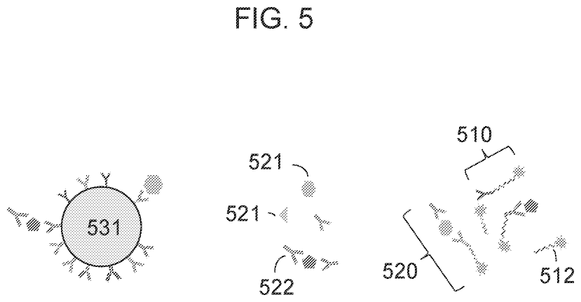

Examples of released sandwich components are shown schematically in FIG. 5 and include, but are not limited to, analyte 521, capture molecule 522, targeting reagent 512, detection reagent 510 and sandwich structures 520. In one aspect, the released sandwich components include labeled targeting reagent, labeled detection reagent, labeled detection reagent with bound analyte and labeled sandwich structures. In one aspect, the released sandwich components include labeled targeting reagent. In one aspect, the amount of labeled targeting reagent bound to its corresponding targeting reagent complement is proportional to the amount of target analyte in a sample.

In one aspect, the sandwich complex is incubated with the release reagent for less than about 1 hour, 45 minutes, 30 minute, 15 minutes, 10 minutes or 5 minutes.

In one aspect, the released sandwich components are separated from the particulate support surface. In one aspect, the particulate support surface is magnetic and the released sandwich components are separated from the particulate support surface by imposition of a magnetic field to remove the particulate support surface from the released sandwich components. In another aspect, the particulate support surface is not magnetic and the released sandwich components are separated from the particulate support surface by centrifugation or filtration.

In one aspect, the method includes a step of amplifying one or more sandwich components after the sandwich components are released from the particulate support surface. In one aspect, the step of amplifying one or more sandwich components includes amplifying an oligonucleotide sequence of the targeting reagent, for example, using polymerase chain reaction (PCR) amplification.

3. Detection

In one aspect, the method includes a detection step in which the presence of a label immobilized on an assay surface is detected. In one aspect, the released detectable portion or released sandwich components are transferred to an assay surface on which a targeting reagent complement is immobilized and the presence of the label immobilized on the assay surface is detected. In one aspect, the released detectable portion or released sandwich component include labeled targeting reagent and are transferred to an assay surface on which a targeting reagent complement is immobilized under conditions in which the labeled targeting reagent specifically binds to its corresponding targeting reagent complement. In one aspect, the presence of the label immobilized on the assay surface is detected.

In one aspect, the assay surface includes a flat surface. In another aspect, the assay surface includes a curved surface. In one aspect, the assay surface includes an assay module, such as an assay plate, slide, cartridge, bead, or chip. As used herein, the term “bead” refers to a support structure that can have any suitable shape, including, but not limited to, spherical, ovoid, cylindrical or any other recognized particle shape with regular or irregular dimensions. In one aspect, the assay surface includes color coded microspheres. In one aspect, the assay surface includes one or more beads on which one or more targeting reagent complements are immobilized.

The assay surface can be made from a variety of suitable materials including polymers, such as polystyrene and polypropylene, ceramics, glass, composite materials, including, for example, carbon-polymer composites such as carbon-based inks. In one aspect, the assay surface includes a carbon-based assay surface.

In one aspect, the assay surface is a plate with more than one well, for example, a “multi-well plate.” Multi-well plates can include any number of wells of any size or shape, arranged in any pattern or configuration. In one aspect, the multi-well plate includes from about 1 to about 10,000 wells. In one aspect, the multi-well assay plates use industry standard formats for the number, size, shape and configuration of the plate and wells. Examples of standard formats include 96-, 384-, 1536- and 9600-well plates, with the wells configured in two-dimensional arrays. Other multi-well formats include single well, two well, six well and twenty-four well and 6144 well plates. In one aspect, the assay surface includes a 96 well-plate.

In one aspect, the released detectable portion or released sandwich components can be transferred to the assay surface without the need for a wash step following the release step. Unbound sandwich components that include a label, such as an ECL label, will not be detected. Upon application of the appropriate voltage waveform, only ECL labeled targeting reagent that is bound to its corresponding targeting reagent complement immobilized on an electrode of the assay surface will be detected. In one aspect, the amount of labeled targeting reagent bound to the targeting reagent complement is proportional to the amount of analyte in the sample.

In one aspect, the released detectable portion or sandwich components are transferred to an assay surface after the particulate support surface is separated from the detectable portion or sandwich components. In one aspect, the released detectable portion or sandwich components are transferred to an assay surface and incubated for at least about 15, about 20, about 25 or about 30 minutes and up to about 30, about 45, about 60 or about 80 minutes to allow the targeting reagent to specifically bind to its corresponding targeting reagent complement.

In one aspect, the assay surface includes an excess of targeting reagent complement such that at least about 90%, 95%, 96%, 97%, 98%, 99% or 100% of the labeled targeting reagent transferred to the assay surface is able to bind to its corresponding targeting reagent complement.

A variety of assay formats are known and can be used in connection with the method described herein. Suitable assay surfaces for use in the methods described herein are known and include surfaces used in binding assays such as those disclosed in International Appl. No. PCT/US2015/030925, published as WO 2015/175856.

In one aspect, the assay surface includes an immobilized targeting reagent complement.

In one aspect, the assay surface includes a two-dimensional patterned array in which a plurality of targeting reagent complements are printed at known locations, referred to as binding domains. In one aspect, the assay surface includes a patterned array of discrete, non-overlapping, addressable binding domains to which targeting reagent complements are immobilized. In one aspect, the targeting reagent complements include an oligonucleotide sequence and the sequence of the targeting reagent complement in each binding domain is known and can be correlated with an appropriate target analyte. In one aspect, all targeting reagent complements in a particular binding domain have the same sequence and the targeting reagent complements in one binding domain have a sequence different from targeting reagent complements in other binding domains.

In one aspect, multiple binding domains are arrayed in orderly rows and columns on a assay surface and the precise location and sequence of each binding domain is recorded in a computer database. In one aspect, the array is arranged in a symmetrical grid pattern. In other aspects, the array is arranged another pattern, including, but not limited to, radially distributed lines, spiral lines, or ordered clusters. In another aspect, each binding domain is positioned on a surface of a microparticle or bead wherein the microparticle or bead is coded to allow for discrimination between different binding domains.

In one aspect, a targeting reagent complement is covalently immobilized on a binding domain on the assay surface. In one aspect, a targeting reagent complement is non-covalently immobilized on a binding domain on the assay surface. In one aspect, multiple distinct binding domains are present for multiplexed measurement of target analytes in a sample. In one aspect, multiple distinct binding domains are present on one or more electrodes for multiplexed measurement of target analytes in a sample. In one aspect, each well includes at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24 or about 25 binding domains. In one aspect, each well includes at least about 7, about 10, about 16, or about 25 binding domains.

In one aspect, the assay surface is a multi-well plate that includes at least about 24, about 96, or about 384 wells and each well includes array of up to 10 binding domains in which different targeting reagent complements are immobilized in discrete binding domains. In a more particular aspect, the assay surface is a 96 well plate in which each well includes an array having up to 10 binding domains. In one aspect, each well of a 96-well plate includes up to 10 binding domains, having up to 10 distinct targeting reagent complements immobilized thereon. In one aspect, each well includes the same patterned array with the same targeting reagent complements. In another aspect, different wells may include a different patterned array of targeting reagent complements.

In one aspect, the assay surface includes one or more electrodes. In one aspect, the assay surface includes one or more working electrodes and one or more counter electrodes. In one aspect, the label is an electrochemiluminescent (ECL) label and the assay surface includes one or more working electrodes and one or more counter electrodes suitable for triggering an electrochemiluminescent emission from a label of an immobilized reaction product. Multiplexed measurement of analytes using electrochemiluminescence is described in U.S. Pat. Nos. 7,842,246 and 6,977,722, the disclosures of which are incorporated herein by reference in their entireties. In one aspect, the assay surface includes one or more binding domains formed on one or more electrodes for use in electrochemical or electrochemiluminescence assays.

In one aspect, the assay surface is multi-well plate that includes at least one electrode. In one aspect, each well of a multi-well assay plate includes at least one electrode. In one aspect, at least one well of the multi-well assay plate includes a working electrode. In another aspect, at least one well of the multi-well assay plate includes a working electrode and a counter electrode. In another aspect, each well of the multi-well assay plate includes a working electrode and a counter electrode. In one aspect, the working electrode is adjacent, but not in electrical contact with the counter electrode.

In one aspect, a targeting reagent complement is covalently immobilized in a binding domain on an electrode on the assay surface. In one aspect, a targeting reagent complement is non-covalently immobilized in a binding domain on an electrode on the assay surface. In one aspect, multiple distinct binding domains are present on one or more electrodes for multiplexed measurement of target analytes in a sample. In one aspect, each well includes at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24 or about 25 binding domains on one or more electrodes. In one aspect, each well includes at least about 7, about 10, about 16, or about 25 binding domains on one or more electrodes.

In one aspect, the electrodes are constructed from a conductive material, including, for example, a metal such as gold, silver, platinum, nickel, steel, iridium, copper, aluminum, a conductive alloy, or combinations thereof. In another aspect, the electrodes include semiconducting materials such as silicon and germanium or semi-conducting films such as indium tin oxide (ITO) and antimony tin oxide (ATO). In another aspect, the electrodes include oxide coated metals, such as aluminum oxide coated aluminum. In one aspect, the electrode includes a carbon-based material. In one aspect, the electrodes include mixtures of materials containing conducting composites, inks, pastes, polymer blends, and metal/non-metal composites, including for example, mixtures of conductive or semi-conductive materials with non-conductive materials. In one aspect, the electrodes include carbon-based materials such as carbon, glassy carbon, carbon black, graphitic carbon, carbon nanotubes, carbon fibrils, graphite, carbon fibers and mixtures thereof. In one aspect, the electrodes include conducting carbon-polymer composites, conducting polymers, or conducting particles dispersed in a matrix, for example, carbon inks, carbon pastes, or metal inks. In one aspect, the working electrode is made of a carbon-polymer composite that includes, for example, conducting carbon particles, such as carbon fibrils, carbon black, or graphitic carbon, dispersed in a matrix, for example, a polymer matrix such as ethylene vinyl acetate (EVA), polystyrene, polyethylene, polyvinyal acetate, polyvinyl chloride, polyvinyl alcohol, acrylonitrile butadiene styrene (ABS), or copolymers of one or more of these polymers.

In one aspect, the working electrode is made of a continuous conducting sheet or a film of one or more conducting materials, which may be extruded, pressed or molded. In another aspect, the working electrode is made of a conducting material deposited or patterned on a substrate, for example, by printing, painting, coating, spin-coating, evaporation, chemical vapor deposition, electrolytic deposition, electroless deposition, photolithography or other electronics microfabrication techniques. In one aspect, the working electrode includes a conductive carbon ink printed on a polymeric support, for example, by ink-jet printing, laser printing, or screen-printing. Carbon inks are known and include materials produced by Acheson Colloids Co. (e.g., Acheson 440B, 423ss, PF407A, PF407C, PM-003A, 30D071, 435A, Electrodag 505SS, and Aquadag™), E. I. Du Pont de Nemours and Co. (e.g., Dupont 7105, 7101, 7102, 7103, 7144, 7082, 7861D, and CB050), Conductive Compounds Inc (e.g., C-100), and Ercon Inc. (e.g., G-451).

In one aspect, the working electrode is a continuous film. In another aspect, the working electrode includes one or more discrete regions or a pattern of discrete regions. Alternately, the working electrode may include more than one connected region. One or more regions of exposed electrode surface on a working electrode can be defined by a patterned insulating layer covering the working electrode, for example, by screen printing a patterned dielectric ink layer over a working electrode, or by adhering a die-cut insulating film. The exposed regions may define the array elements of arrays of reagents printed on the working electrode and may take on array shapes and patterns as described above. In one aspect, the insulating layer defines a series of circular regions (or “spots”) of exposed working electrode surface.

A counter electrode may have one or more of the properties described above generally for working electrodes. In one aspect, the working and counter electrodes are constructed from the same material. In another aspect, the working and counter electrodes are not constructed from the same material, for example, the working electrode may be a carbon electrode and the counter electrode may be a metal electrode.

In one aspect, a targeting reagent complement is immobilized on an electrode by passive adsorption. In another aspect, a targeting reagent complement is covalently immobilized on the electrodes. In one aspect, the electrodes are derivatized or modified, for example, to immobilize reagents such as targeting reagent complements on the surface of the electrodes. In one aspect, the electrode is modified by chemical or mechanical treatment to improve the immobilization of reagents, for example, to introduce functional groups for immobilization of reagents or to enhance its adsorptive properties. Examples of functional groups that can be introduced include, but are not limited, to carboxylic acid (COOH), hydroxy (OH), amino (NH₂), activated carboxyls (e.g., N-hydroxy succinimide (NHS)-esters), poly-(ethylene glycols), thiols, alkyl ((CH₂)ₙ) groups, or combinations thereof). In one aspect, one or more reagents, for example, one or more targeting reagent complements, are immobilized by either covalent or non-covalent means to a carbon-containing electrode, for example, carbon black, fibrils, or carbon dispersed in another material. It has been found that targeting reagent complements having thiol groups can bind covalently to carbon-containing electrodes, for example to screen-printed carbon ink electrodes, without having to first deposit an additional thiol-reactive layer such as a protein layer or a chemical cross-linking layer. In one aspect, methods are provided for direct attachment of targeting reagent complements having thiol groups, such as thiol-modified oligonucleotides, to electrodes which provide simple, robust, efficient and reproducible processes for forming arrays on electrodes. In one aspect, a targeting reagent complement having a thiol group is directly immobilized on a carbon-containing electrode, such as a screen-printed carbon ink electrode, through reaction of the thiols with the electrode, without first adding a thiol-reactive layer to the electrode.

In one aspect, one or more binding domains are located on one or more electrodes of the assay surface. In one aspect, detecting, identifying or quantifying a target analyte includes applying a voltage waveform to one or more electrodes to stimulate the labeled sandwich components on the assay surface, including, for example, labeled targeting reagent, to produce an electrochemical or luminescent signal. In one aspect, detecting, identifying or quantifying includes measuring an electrochemiluminescent signal and correlating the signal with the presence or an amount of target analyte in a sample. In one aspect, the intensity of the emitted light is proportional to the amount target in the sample such that the emitted light can provide a quantitative determination of the amount of target analyte in the sample.

In one aspect, one or more binding domains are formed by collecting beads coated with targeting reagent complement onto the electrode surface. In one aspect, the beads are magnetic or paramagnetic and the beads are collected on the electrode through the use of a magnetic field.

In one aspect, the label includes an amplification primer and detection includes contacting the primer under conditions in which an amplification product is generated. In one aspect, the label includes a rolling circle amplification primer (“RCA primer”) and detection includes contacting the RCA primer with a circular template under conditions in which a rolling circle amplification product (“RCA product”) is generated. In one aspect, the RCA primer includes a nucleotide sequence that is complementary to a nucleotide sequence of the circular template. In one aspect, the method includes contacting the RCA primer with the circular template in the presence of a DNA polymerase under conditions in which the DNA polymerase catalyzes replication of the circular template to form the RCA product. In one aspect, the RCA product includes tandem repeats of a nucleotide sequence complementary to the nucleotide sequence of the circular template. In one aspect, the RCA product includes at least about 100, or up to about 1000, about 5000 or about 10,000 tandem repeats of a nucleotide sequence complementary to the nucleotide sequence of the circular template. Reagents for rolling circle amplification are described in U.S. Patent Publication No. 2004/0191784 (UNIVERSAL REAGENTS FOR ROLLING CIRCLE AMPLIFICATION AND METHODS OF USE), the disclosure of which is incorporated by reference herein in its entirety. Methods for multiplex detection of molecules of interest using rolling circle amplification are described in PCT Publication No. 1997/019193 (UNIMOLECULAR SEGMENT AMPLIFICATION AND DETECTION), the disclosure of which is incorporated by reference herein in its entirety.

In one aspect, the RCA product is detected using a labeled detection probe that is capable of hybridizing to the RCA product. In one aspect, the labeled detection probe includes a nucleotide sequence that is complementary to a nucleotide sequence of the RCA product. In one aspect, the labeled detection probe includes a nucleotide sequence that is at least about 15, about 20, about 25, about 30, about 35, about 40, about 45 or about 50 nucleotides in length and up to about 50, about 60, about 70, about 80, about 90 or about 100 nucleotides in length. In one aspect, the detection probe is labeled with an ECL label. In one aspect, the detection probe is labeled with a fluorescent label.

In one aspect, each detection probe is labeled with a combination of at least two fluorescent labels in a ratio that allows probes to be discriminated from each other based on the ratio of the fluorescent labels. In one aspect, the fluorescent labels are conjugated to the detection probe at one or more locations to achieve the desired labeling ratio. In one aspect, the detection probes are distinguished by variations in the labeling intensity. In one aspect, two fluorescent labels are used to label the detection probes. In one aspect, three fluorescent labels are used to label the detection probes. In one aspect, up to 500 uniquely encoded detection probes can be generated using two or three fluorescent labels.

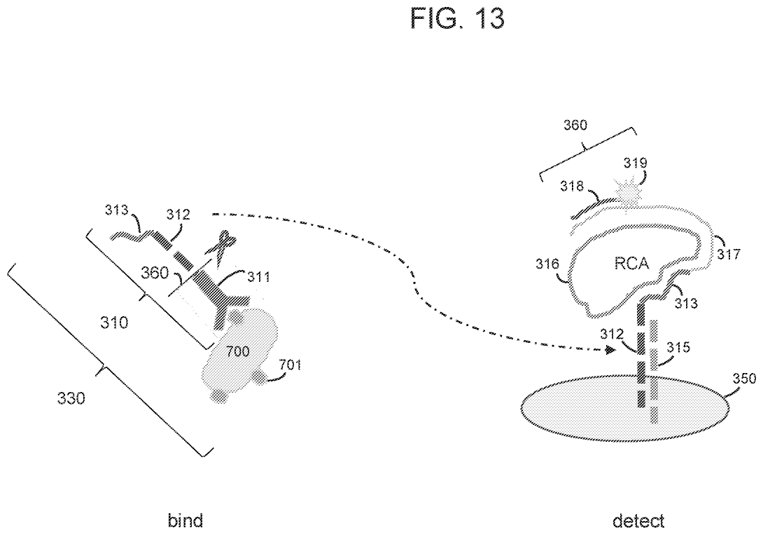

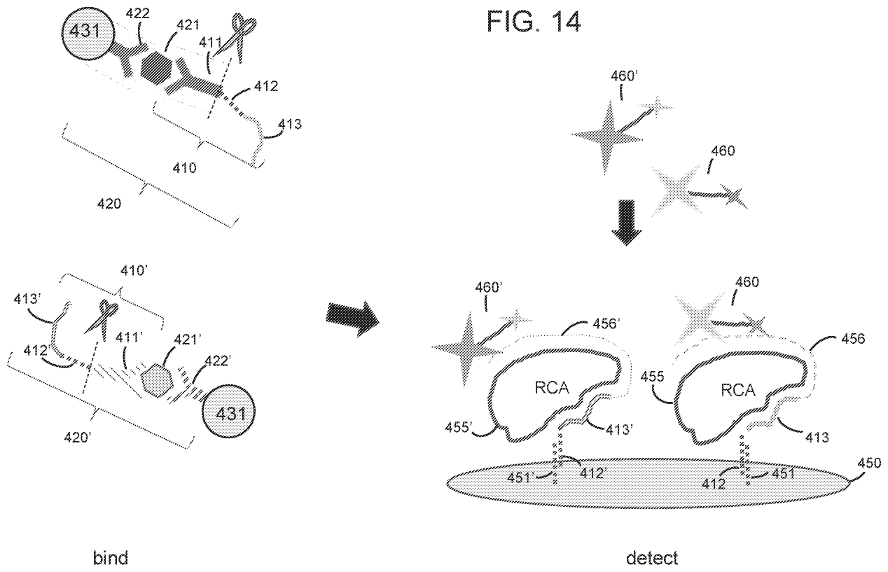

FIG. 13 is a schematic of an assay in which the label includes an RCA primer. In one aspect, the method includes contacting a cell or tissue 700 with a detection reagent 310 under conditions in which the analyte binding portion 311 of the detection reagent 310 specifically binds to the target analyte 701 to form a binding complex 330. In one aspect, the detection reagent 310 includes an analyte binding portion 311, a targeting reagent 312 and a label 313. In one aspect, the analyte binding portion 311 of the detection reagent 310 is an antibody and the label 313 includes an RCA primer. In one aspect, the analyte binding portion 311 of the detection reagent 310 is an antibody, the targeting reagent 312 is an oligonucleotide and the label 313 includes an RCA primer. In one aspect, the detection reagent 310 includes a digestible linker 360. In one aspect, the targeting reagent 312 and label 313 are attached to the analyte binding portion 311 through a digestible linker 360. In one aspect, the targeting reagent 312 includes an oligonucleotide and the analyte binding portion 311 includes a peptide sequence. In one aspect, a detectable portion of the detection reagent 310 that includes the targeting reagent 312 and label 313 is released from the binding complex 330. In one aspect, the detectable portion of the detection reagent 310 is released by contacting the binding complex with a restriction enzyme. In one aspect, the detectable portion of the detection reagent 310 is released by contacting the binding complex with a protease. In one aspect, the detectable portion is transferred to an assay surface 360 on which a targeting reagent complement 315 is immobilized under conditions in which the targeting reagent 312 binds to the targeting reagent complement 315. In one aspect, the assay surface to which the detectable portion is immobilized is contacted with a circular RCA template that binds to the RCA primer 313 under conditions in which an RCA product 317 is generated. In one aspect, the RCA product 317 is detected using a labeled detection probe 360 that includes a detectable label 319 and a nucleotide sequence 318 that is complementary to a nucleotide sequence of the RCA product 317. In one aspect, the RCA product 317 includes tandem repeats of a nucleotide sequence complementary to the labeled detection probe 360 (not shown).

In one aspect, more than one analyte in a sample is detected in a multiplex assay. In one aspect, the multiplex assay includes a first detection reagent with a first analyte binding portion specific for a first analyte and a first label, wherein the first analyte binding portion includes a first oligonucleotide with a first sequence; and a second detection reagent with a second analyte binding portion specific for a second analyte and a second label, wherein the second analyte binding portion includes a second oligonucleotide with a second sequence and a second label, etc., wherein the assay includes a detection reagent specific for each target analyte in the sample.

FIG. 14 is a schematic of a multiplex assay in which more than one analyte in a sample is assayed. In one aspect, the multiplex assay includes a first detection reagent 410 with a first analyte binding portion 411 specific for a first target analyte 421, a first targeting reagent 412 and a first label 413. In one aspect, the multiplex assay includes a second detection reagent 410′ with a second analyte binding portion 411′ specific for a second target analyte 421′, a second targeting reagent 412′ and a second label 413′. In one aspect, the first and second analyte binding portions 411, 411′ include a peptide sequence. In one aspect, the first and second analyte binding portions 411, 411′ are antibodies. In one aspect, the first and second targeting reagents 412, 412′ are oligonucleotides. In one aspect, the first and second labels 413, 413′ include an RCA primer. In one aspect, the first and second analyte binding portions 411, 411′ are different antibodies and specifically bind the different target antigens 421, 421′ or different epitopes of the same target antigen 421, 421′. In one aspect, the first and second RCA primer sequences 413, 413′ have different sequences. In one aspect, the first and second detection reagents 410, 410′ are added to the same sample.

In one aspect, a detectable portion that includes a targeting reagent 412, 412′ and RCA primer 413, 413′ is released from each binding complex 410, 410′ and transferred to an assay surface 450. In one aspect, the assay surface 450 includes one or more targeting reagent complements 451, 451′ to which the detectable portions bind. In one aspect, the assay surface 450 includes a unique targeting reagent complement 451, 451′ for each targeting reagent 412, 412′ in the assay. In one aspect, the targeting reagents 412, 412′ and the targeting reagent complements 451, 451 include oligonucleotides. In one aspect, the targeting reagents 412, 412′ and the targeting reagent complements 451, 451 include single stranded oligonucleotides. In one aspect, the first targeting reagent 412 and the first targeting reagent complement 451 have complementary sequences and the second targeting reagent 412 and the second targeting reagent complement 451 have complementary sequences.

In one aspect, the assay surface 450 is contacted with an RCA template 455, 455′ under conditions in which an RCA product 456, 456′ is generated. In one aspect, an RCA template 455, 455′ with a different nucleotide sequence is provided for each RCA primer 413, 413′ to generate a unique RCA product 456, 456′ associated with each targeting reagent 413, 413′. In one aspect, each unique RCA product 456, 456′ includes a unique sequence to which a corresponding labeled detection probe 460, 460′ specifically binds. In one aspect, each labeled detection probe 460, 460′ includes a unique fluorescent label or a unique ratio of fluorescent labels such that the RCA product can be distinguished based on the fluorescent signature of the corresponding labeled detection probe 460, 460′.

In one aspect, a multiplex assay is provided in which more than one target analyte in more than one sample is assayed. In one aspect, the assay includes first and second detection reagents 410, 410′ that each include first and second analyte binding portions 411, 411′. In one aspect, the first and second analyte binding portions 411, 411′ include the same antibody that specifically binds to the same target antigen. In one aspect, the first detection reagent 410 includes a first label 413 that is a first RCA primer with a first primer sequence and the second detection reagent 410 includes a second label 413 that is a second RCA primer with a second primer sequence. In one aspect, the first and second RCA primer sequences are different. In one aspect, a first sample is contacted with the first detection reagent 410 and a second sample is contacted with the second detection reagent 410′ wherein the first and second samples include the same target analyte. In one aspect, a coated particle 431 on which a capture reagent 422 is immobilized is added to the first and second samples. In one aspect, first and second sandwich complexes 420, 420′ are formed in the respective reaction mixtures. In one aspect, a first detectable portion is released from the first sandwich complex 420 and a second detectable portion is released from the second sandwich complex 420′. In one aspect, the first and second detectable portions 420, 420′ are separated from the particulate support surface 431 and transferred to an assay surface 450. In one aspect, the mixtures containing the first and second detectable complexes are combined and transferred to the same assay surface 450. In one aspect, the assay surface 450 is contacted with a first RCA template 455 specific for the first RCA primer 413 and a second RCA template 455′ specific for the second RCA primer 413′. In one aspect, each RCA template 455, 455′ is associated with a unique corresponding detection probe 460, 460′ that has a unique fluorescence signature such that the amount of target analyte in each sample can be determined.

### G. Kits

In one aspect, a system or kit is provided for conducting assays for detecting one or more analytes in a sample. In one aspect, a system or kit is provided for multiplexed assays for detecting a plurality of analytes in a sample. In one aspect, the kit includes an analyte binding system and a detection system. In one aspect, the kit includes a release reagent.

In one aspect, the analyte binding system includes a detection reagent that includes an analyte binding portion, a targeting reagent and a detectable label. In one aspect, the analyte binding system includes one or more of the following: a particulate support surface, a capture molecule, and a detection reagent or components thereof. In one aspect, the particulate support surface is magnetic or paramagnetic. In one aspect, the particulate support surface is coated with a member of a binding pair. In one aspect, the particulate support surface is coated with avidin, streptavidin or biotin. In one aspect, the particulate support surface includes a carboxyl or amine coating. In one aspect, the kit includes an immobilization reagent suitable for immobilizing a capture molecule on the particulate support surface. In one aspect, the immobilization reagent includes avidin, streptavidin or biotin.

In one aspect, the analyte binding system includes a component of a detection reagent. In one aspect, the analyte binding system includes a label and a targeting reagent. In one aspect, the label is attached directly to the detection reagent. In another aspect, the label is attached to the detection reagent through a linker. In one aspect, the label includes a radioactive, fluorescent, chemiluminescent, electrochemiluminescent, light absorbing, light scattering, electrochemical, magnetic or enzymatic label. In one aspect, the label includes an electrochemiluminescent (ECL) label. In one aspect, the label includes an organometallic complex that includes a transition metal. In one aspect, the transition metal includes ruthenium. In one aspect, the label is a MSD SULFO-TAG™ label. In one aspect, the system or kit includes a detection reagent that includes a carrier protein conjugated to multiple labeled targeting reagents.

In one aspect, the kit includes one or more components for rolling circle amplification (RCA). In one aspect, kit includes a rolling circle amplification primer (“RCA primer”) and a circular RCA template. In one aspect, the RCA primer includes a nucleotide sequence that is complementary to a nucleotide sequence of the circular template. In one aspect, the kit includes a DNA polymerase. In one aspect, the RCA primer includes a reactive group, such as a thiol (—SH) or hapten, for example, biotin, avidin or streptavidin, to facilitate conjugation of the RCA primer to a detection reagent. In one aspect, the kit includes a labeled detection probe that is capable of hybridizing to an RCA product. In one aspect, the labeled detection probe includes a nucleotide sequence that is complementary to a nucleotide sequence of the RCA product. In one aspect, the labeled detection probe includes a nucleotide sequence that is at least about 15, about 20, about 25, about 30, about 35, about 40, about 45 or about 50 nucleotides in length and up to about 50, about 60, about 70, about 80, about 90 or about 100 nucleotides in length. In one aspect, the detection probe is labeled with an ECL label. In one aspect, the detection probe is labeled with a fluorescent label. In one aspect, the detection probe is labeled with a combination of at least two fluorescent labels in a ratio that allows the probes to be discriminated from each other. In one aspect, the fluorescent labels are conjugated to the detection probe at one or more locations to achieve the desired label labeling ratio. In one aspect, the kit includes two or more detection probes separately labeled with a single fluorescent label such that the detection probes can be combined to achieve the desired labeling ratio. In one aspect, two fluorescent labels are used to label the detection probes. In one aspect, three fluorescent labels are used to label the detection probes. In one aspect, up to 500 uniquely encoded detection probes can be generated using two or three fluorescent labels.

In one aspect, kit includes a release reagent. In one aspect, the release agent is configured to release a detectable portion, including labeled targeting reagent, from a binding complex. In one aspect, the release agent is configured release one or more sandwich components, including labeled targeting reagent, from a sandwich complex. In one aspect, the release reagent includes a compound that denatures or digests peptide sequences but leaves oligonucleotide sequences intact. In one aspect, the release reagent includes a protease. In one aspect, the protease is selected from: a serine protease, cysteine protease, threonine protease, aspartic protease, glutamic protease, and metallo protease. In one aspect, the protease includes a serine protease. In one aspect, the protease includes proteinase K. In one aspect, the release reagent includes an endopeptidase such as pepsin or trypsin. In one aspect, the release reagent includes a reducing reagent such as ß-mercaptoethanol (BME) or dithiothritol (DTT). In one aspect, the release reagent includes a restriction enzyme.

In one aspect, the detection system includes an assay surface. In one aspect, the detection system includes a targeting reagent complement. In one aspect, the detection system includes an assay surface and a targeting reagent complement. In one aspect, the assay surface includes a multi-well plate. In one aspect, the assay surface includes a plurality of particles. In one aspect, the assay surface includes a plurality of discrete binding domains. In one aspect, a targeting reagent complement is immobilized on the assay surface. In one aspect, a targeting reagent complement is immobilized in a discrete binding domain on the assay surface. In one aspect, the kit includes in a separate vial, container, or compartment, a targeting reagent complement that can be immobilized on the assay surface. In one aspect, the kit includes, in a separate vial, container or compartment, a targeting reagent complement that can be immobilized on one or more discrete binding domains on the assay surface.

In one aspect, the kit includes one or more system components, for example, a support surface and one or more containers, vessels or compartments that include one or more reagents.

In one aspect, the kit includes one or more reagents that are stored in a dry state and may further include or be supplied with desiccant materials for maintaining the reagents in a dry state. Assay devices preloaded with the reagents can improve the speed and reduce the complexity of assay measurements while maintaining stability during storage. The dried assay reagents may be any assay reagent that can be dried and then reconstituted prior to use. These include, but are not limited to, capture molecules, targeting reagents, targeting reagent complements, enzymes, enzyme substrates, labels and other compounds that may be used to detect a target analyte. In one aspect, the kit includes one or more reagents or substances that are not directly involved in analyte binding or detection but play an auxiliary role including, but not limited to, blocking agents, stabilizing agents, detergents, salts, pH buffers, preservatives, etc. Reagents may be present in free form or supported on solid phases including the surfaces of compartments (e.g., chambers, channels, flow cells, wells, etc.) in the assay modules or the surfaces of colloids, beads, or other particulate supports.

### H. Incorporation by Reference

All references cited herein, including patents, patent applications, papers, text books and the like, and the references cited therein, to the extent that they are not already, are hereby incorporated herein by reference in their entirety.

### WORKING EXAMPLES

### Example 1. Multiplexed Assay in which Capture Molecule is Immobilized on Particulate Support Surface

Streptavidin coated magnetic microparticles can be incubated with a solution containing biotinylated capture antibodies, and washed to immobilize the capture antibodies on the microparticles. It is noted that the microparticles with the immobilized capture antibodies can be prepared in advance.

A sample that contains or is suspected of containing one or more analytes of interest is added to the capture antibody-coated microparticles, incubated and washed.

A labeled detection antibody that includes an analyte binding antibody portion, a targeting reagent that includes an oligonucleotide sequence that is complementary to an oligonucleotide sequence of a targeting reagent complement immobilized on an assay surface and an ECL label is then added to the sample and incubated to form a sandwich complex that includes the microparticle and one or more sandwich components that include the capture antibody, bound analyte, and labeled detection antibody. The sandwich complex can be washed to remove unbound detection antibody.

The washed sandwich complex is then contacted with a release reagent that includes proteinase K and incubated to release the sandwich components from the magnetic microparticle. The magnetic microparticle can be separated from the supernatant containing the released sandwich components by imposition of a magnetic field.

The supernatant that contains the released sandwich components is then transferred to a 96-well assay plate on which targeting reagent complement is immobilized on one or more working electrodes in discrete binding domains and incubated to allow the labeled targeting reagent to bind to its corresponding targeting reagent complement. Advantageously, the supernatant can be transferred to the assay surface without the need for an additional wash step because unbound sandwich components and label in the matrix will not be illuminated.

A voltage waveform is applied to one or more electrodes to stimulate the bound and labeled sandwich components on the assay surface to produce an electrochemical or luminescent signal which can be measured and correlated with the presence or an amount of target analyte in the sample.

### Example 2. Multiplexed Assay in which Sandwich Structure is Immobilized on Particulate Support Surface

A sample that contains or may contain one or more target analytes of interest is combined with one or more capture antibodies and one or more labeled detection antibodies and incubated to allow sandwich structures to form. The capture antibodies specifically bind one or more target analytes and include biotin as an immobilization reagent. The labeled detection antibodies specifically bind one or more target analytes and include an analyte binding antibody portion, a targeting reagent that includes an oligonucleotide sequence that is complementary to an oligonucleotide sequence of a targeting reagent complement immobilized on an assay surface, and an ECL label.

The biotinylated sandwich structure is incubated with commercially available streptavidin coated magnetic microparticles to immobilize the sandwich structures on the microparticles to form sandwich complexes which are then washed to remove unbound sandwich structures.

The washed sandwich complex is then contacted with a release reagent and assayed as described in Example 1.

### Example 3. Cell Based Assays with Oligonucleotide Coded Antibodies

A sample that contains or may contain one or more surface markers of interest, for example, a population of cells, is incubated with up to 10 different detection reagents that each include (i) an analyte binding portion that includes an antibody that specifically binds to a surface marker of interest, and (ii) a labeled oligonucleotide targeting reagent that includes a nucleotide sequence that is complementary to the nucleotide sequence of a targeting reagent complement that is immobilized on a MSD Multi-Array plate. The label includes an ECL label, biotin, and/or a primer for rolling circle amplification. The oligonucleotide targeting reagent can be conjugated to the antibody analyte binding portion through a thiol (—SH) group or through a binding pair, such as biotin and avidin/streptavidin.

Cells in suspension can be captured onto a particulate support surface using known immunocapture methods and incubated with the detection reagent(s) under conditions in which the antibody analyte binding portion of each detection reagent specifically binds to its corresponding surface marker of interest. After excess reagent is removed by washing, the labeled oligonucleotide targeting reagent portion of the detection reagent(s) on the captured cells is released, for example, using trypsin or other chaotropic agents or using enzymatic (i.e. nucleases) or photochemical methods.

An assay plate on which up to 10 targeting reagent complements are immobilized is contacted with the eluted oligonucleotide targeting reagent(s) and the presence of the targeting reagent is detected, for example, by ECL detection, fluorescence detection or by RCA amplification.

1. A method of conducting an assay for an analyte in a sample, the method comprising:
(a) contacting the sample with a detection reagent comprising an analyte binding portion, a targeting reagent, and a label to form a binding complex comprising analyte and the detection reagent;
(b) removing unbound detection reagent;
(c) contacting the binding complex with a release reagent to release a detectable portion of the binding complex, wherein the detectable portion comprises the targeting reagent and the label;
(d) transferring the detectable portion released from the binding complex to an assay surface on which a targeting reagent complement is immobilized under conditions in which the targeting reagent hybridizes to the targeting reagent complement; and
(e) detecting a presence of the label immobilized on the assay surface, wherein an amount of label is proportional to an amount of analyte in the sample.

2. The method of claim 1, wherein the sample comprises an environmental or clinical sample.

3. The method of claim 1, wherein the analyte is in suspension.

4. The method of claim 1, wherein the analyte is not in suspension.

5. The method of claim 1, wherein the sample comprises a protein, polypeptide, oligonucleotide, lipid, steroid, carbohydrate, porphyrin, alkaloid, virus, microorganism, cell, tissue, organ or subcellular particle.

6. The method of claim 1, wherein the sample comprises a tissue, cell or organism.

7. The method of claim 1, wherein the sample is immobilized on a surface.

8. The method of claim 1, wherein the sample comprises a live tissue sample or a fixed tissue sample.

9. The method of claim 1, wherein the analyte is an antibody, enzyme, receptor, hormone or structural protein.

10. The method of claim 1, wherein the analyte comprises single stranded DNA, double stranded DNA, single stranded RNA or double stranded RNA.

11. The method of claim 1, wherein the analyte comprises miRNA, therapeutic RNA, mRNA, or an RNA virus.

12. The method of claim 1, wherein the analyte is associated with a tissue, cell or organism.

13. The method of claim 12, wherein the analyte comprises a cell surface marker.

14. The method of claim 13, wherein the cell surface marker comprises a protein, glycoprotein, enzyme or carbohydrate.

15. The method of claim 13, wherein the cell surface marker is an integral membrane protein, a transmembrane protein or a peripheral membrane protein.

16. The method of any of claims 1 to 15, wherein the analyte binding portion of the detection reagent is selected from: an oligonucleotide comprising an analyte binding nucleic acid sequence that is complementary to a nucleic acid sequence of the analyte, an antibody that specifically binds an analyte, a receptor that specifically binds an analyte comprising a ligand, or a ligand that specifically binds an analyte comprising a receptor.

17. The method of any of claims 1 to 16, wherein the analyte binding portion of the detection reagent comprises an oligonucleotide comprising an analyte binding nucleic acid sequence that is complementary to a nucleic acid sequence of the analyte.

18. The method of claim 1, wherein the sample is contacted with the detection reagent, a capture molecule and a particulate support surface in (a).

19. The method of claim 18, wherein the binding complex in (a) comprises a sandwich complex comprising capture molecule, bound analyte and detection reagent that is immobilized on the particulate support surface.

20. The method of claim 19, wherein (c) comprises contacting the sandwich complex with the release reagent and the detectable portion comprises a sandwich component that is released from the particulate support surface.

21. The method of claim 20, wherein the sandwich component released from the particulate support surface is selected from labeled targeting reagent, labeled detection reagent, labeled detection reagent with bound analyte and labeled sandwich structures.

22. The method of claim 20 or 21, wherein (d) comprises transferring the released sandwich components to the assay surface.

23. The method of any of claims 1 to 16, wherein the analyte binding portion of the detection reagent comprises a protein.

24. The method of claim 23, wherein the analyte binding portion of the detection reagent comprises protein selected from an antibody or receptor, or a portion thereof, that specifically binds an analyte.

25. The method of claim 24, wherein the analyte binding portion of the detection reagent comprises an antibody or an antigen binding antibody fragment that specifically binds the analyte.

26. A method of conducting an assay for an analyte in a sample, the method comprising:
(a) in one or more steps, combining the sample with a particulate support surface, a capture molecule and a detection reagent, wherein the detection reagent comprises an analyte binding portion, a targeting reagent and a label, to form a sandwich complex comprising the particulate support surface on which a sandwich structure comprising capture molecule, bound analyte and detection reagent are immobilized;
(b) removing unbound detection reagent;
(c) contacting the sandwich complex with a release reagent to release sandwich components from the particulate support surface;
(d) transferring the released sandwich components to an assay surface on which a targeting reagent complement is immobilized; and
(e) detecting a presence of the label on the assay surface, wherein an amount of label is proportional to an amount of analyte in the sample.

27. The method of claim 26, wherein step (a) comprises:
(i) combining the sample with a coated particle comprising a particulate support surface on which a capture molecule is immobilized;
(ii) allowing analytes in the sample to bind to the capture molecules to form an analyte coated particle that comprises the coated particle and bound analyte;
(iii) washing the analyte coated particle to remove unbound analyte; and
(iv) contacting the washed analyte coated particle with a detection reagent comprising an analyte binding portion, a targeting reagent and a label to form a sandwich complex that comprises the particulate support surface and sandwich components.

28. The method of claim 26, wherein step (a) comprises:
(i) combining the sample with a capture molecule and a detection reagent;
(ii) allowing analytes in the sample to bind to the capture molecules and detection reagents to form a sandwich structure that comprises the capture molecule, the analyte and the detection reagent; and
(iii) contacting a particulate support surface with the sandwich structure and immobilizing the sandwich structure on the particulate support to form a sandwich complex.

29. The method of any of claims 26 to 28, wherein the analyte binding portion of the detection reagent comprises a nucleotide sequence that is complementary to a nucleotide sequence of the analyte.

30. The method of any one of claims 26 to 29, wherein the capture molecule comprises an immobilization reagent.

31. The method of claim 30, wherein the particulate support surface is coated with avidin or streptavidin and the immobilization reagent comprises biotin.

32. The method of any of claims 26 to 31, wherein the particulate support surface comprises a magnetic particle.

33. The method of any of claims 26 to 32, wherein the capture molecule immobilized on the particulate support surface is selected from: a capture oligonucleotide with a capture nucleic acid sequence that is complementary to a first nucleic acid sequence of the analyte, a capture antibody or antigen binding antibody fragment that specifically binds a first portion of an analyte, a capture receptor or ligand binding receptor fragment that specifically binds a first portion of an analyte comprising a ligand, or a capture ligand that specifically binds a first analyte comprising a receptor.

34. The method of any of claims 26 to 33, wherein each particulate support surface comprises capture molecules specific for one analyte.

35. The method of any of claims 26 to 34, wherein each particulate support surface comprises two or more different capture molecules specific for different analytes.

36. A method of conducting an assay for an immobilized analyte, the method comprising:
(a) contacting a surface with a sample and a detection reagent comprising an analyte binding portion comprising an antibody or an antigen binding antibody fragment, a targeting reagent comprising an oligonucleotide, and a label to form a binding complex comprising bound analyte and the detection reagent;
(b) removing unbound detection reagent;
(c) contacting the binding complex with a release reagent to release a detectable portion of the binding complex, wherein the detectable portion comprises the targeting reagent and the label;
(d) transferring the detectable portion released from the binding complex to an assay surface on which a targeting reagent complements is immobilized; and
(e) detecting presence of the label immobilized on the assay surface, wherein an amount of label is proportional to an amount of analyte in the sample.

37. The method of claim 36, wherein the sample comprises a tissue, cell or organism.

38. The method of claim 36, wherein the sample comprises a live tissue sample or a fixed tissue sample.

39. The method of claim 36, wherein the analyte is associated with a tissue, cell or organism.

40. The method of claim 39, wherein the analyte comprises a cell surface marker.

41. The method of claim 40, wherein the cell surface marker comprises a protein, glycoprotein, enzyme or carbohydrate.

42. The method of claim 41, wherein the cell surface marker comprises an integral membrane protein, a transmembrane protein, or a peripheral membrane protein.

43. The method of any of claims 1 to 42, wherein the targeting reagent comprises a single stranded oligonucleotide.

44. The method of claim 43, wherein the targeting reagent comprises a nucleotide sequence that is complementary to a nucleotide sequence of the targeting reagent complement.

45. The method of any of claims 1 to 44, wherein the targeting reagent is attached to the analyte binding portion of the detection reagent via a digestible linker.

46. The method of claim 45, wherein the digestible linker comprises a disulfide bond.

47. The method of claim 46, wherein the release reagent comprises a reducing reagent.

48. The method of claim 47, wherein the reducing agent is selected from β-mercaptoethanol (BME) and dithiothritol (DTT).

49. The method of claim 45, wherein the digestible linker comprises a peptide.

50. The method of claim 45, wherein the digestible linker comprises a protein.

51. The method of claim 45, wherein the release reagent comprises a protease.

52. The method of claim 51, wherein the protease is selected from: a serine protease, cysteine protease, threonine protease, aspartic protease, glutamic protease, and metallo protease.

53. The method of claim 51 or 52, wherein the protease comprises a serine protease

54. The method of claim 51 or 52, wherein the protease comprises proteinase K.

55. The method of claim 45, wherein the digestible linker comprises an oligonucleotide.

56. The method of claim 55, wherein the digestible linker comprises a restriction site.

57. The method of claim 56, wherein the release reagent comprises a restriction enzyme.

58. The method of claim 26, wherein one or more components of the sandwich complex comprise a restriction site.

59, The method of claim 58, wherein one or more of the capture molecule, the detection reagent, the analyte binding portion, and/or the targeting reagent comprises a restriction site.

60. The method of any of claims 1 to 60, wherein the assay surface comprises a carbon-based electrode.

61. The method of claim 60, wherein the carbon-based electrode comprises a carbon ink electrode.

62. The method of any of claims 1 to 61, wherein the assay surface comprises a multi-well plate.

63. The method of any of claims 1 to 62, wherein the targeting reagent complement is immobilized in a binding domain on the assay surface.

64. The method of claim 62, wherein the multi-well plate comprises a carbon-based electrode.

65. The method of claim 64, wherein the multi-well plate comprises an electrode in a well of the multi-well plate.

66. The method of claim 65, wherein the targeting reagent complement is immobilized on a binding domain on the electrode in the well of the multi-well plate.

67. The method of any of claims 1 to 66, wherein the label comprises an electrochemiluminescent label.

68. The method of claim 67, wherein the label comprises an organometallic complex comprising a transition metal.

69. The method of claim 68, wherein the transition metal comprises ruthenium.

70. The method of any of claims 67 to 69, wherein the label comprises a MSD SULFO-TAG™ label.

71. The method of any of claims 1 to 70, further comprising amplifying the detectable portion after the detectable portion is released from the binding complex.

72. The method of claim 71, wherein amplifying the detectable portion comprises PCR amplification of an oligonucleotide sequence of the targeting reagent.

73. The method of any of claims 1 to 66, wherein the label comprises a primer for rolling circle amplification (RCA primer).

74. The method of claim 73, wherein detecting the presence of the label immobilized on the assay surface comprises contacting the assay surface with a template for rolling circle amplification (RCA template) and generating a rolling circle amplification product (RCA product).

75. The method of claim 74, wherein detecting the presence of the label immobilized on the assay surface comprises contacting the RCA product with a detection probe.

76. The method of claim 75, wherein the detection probe comprises a label and nucleotide sequence that is complementary to a nucleotide sequence of the RCA product.

77. The method of claim 76, wherein the label comprises a fluorescent label.

78. The method of claim 76, wherein the detection probe comprises more than one type of fluorescent label.

79. The method of claim 78, wherein the detection probe has a fluorescent signature based on the ratio between the fluorescent labels.

80. The method of any of claims 1 to 79, wherein the detection reagent is conjugated to a carrier protein comprising multiple labeled targeting reagents.

81. The method of claim 80, wherein the carrier protein is digested with a digestion reagent after the sandwich components are released from the particulate support surface to release the labeled targeting reagent.

82. The method of claim 81, wherein the digestion reagent comprises an enzyme.

83. The method of claim 82, wherein the digestion reagent comprises a protease.

84. The method of claim 81, wherein the labeled targeting reagent is conjugated to the carrier protein through a disulfide bond.

85. The method of claim 84, wherein the digestion reagent comprises a reducing agent.

86. The method of claim 85, wherein the reducing agent is selected from β-mercaptoethanol (BME) and dithiothritol (DTT).

87. A method of conducting an assay for an analyte in a sample, the method comprising:
(a) contacting the sample with a detection reagent comprising an analyte binding portion, a targeting reagent, and a label comprising a primer for rolling circle amplification (RCA primer) to form a binding complex comprising bound analyte and the detection reagent;
(b) removing unbound detection reagent;
(c) contacting the binding complex with a release reagent to release a detectable portion of the binding complex, wherein the detectable portion comprises the targeting reagent and the label;
(d) transferring the detectable portion released from the binding complex to an assay surface on which a targeting reagent complement is immobilized under conditions in which the targeting reagent hybridizes to the targeting reagent complement;
(e) contacting the assay surface with a template for rolling circle amplification (RCA template) under conditions in which a rolling circle amplification product (RCA product) is generated;
(f) contacting the RCA product with a labeled detection probe comprising a nucleotide sequence that is complementary to a sequence of the RCA product; and
(g) detecting presence of the labeled detection probe.

88. The method of claim 87, wherein the sample comprises a plurality of target analytes.

89. The method of claim 88, wherein the method comprises contacting the sample with a unique detection reagent for each target analyte in the sample.

90. The method of claim 89, wherein each unique detection reagent comprises a unique RCA primer.

91. The method of claim 90, wherein the assay surface is contacted with a unique RCA template corresponding to each unique RCA primer to form a unique RCA product.

92. The method of claim 91, wherein each unique RCA product is contacted with a unique labeled detection probe having a unique fluorescent signature.

93. The method of claim 92, wherein the each labeled detection probe comprises a unique fluorescent label.

94. The method of claim 92, wherein each labeled detection probe comprises a unique ratio of fluorescent labels.

95. A system or kit for conducting an assay to detect an analyte in a sample, the kit comprising:
(a) an analyte binding system comprising a detection reagent comprising an analyte binding portion, a targeting reagent and a detectable label;
(b) a release reagent; and
(c) a detection system comprising an assay surface and a targeting reagent complement.

96. The system or kit of claim 95, wherein the analyte binding system further comprises a particulate support surface and a capture molecule.

97. The system or kit of claim 96, wherein the particulate support surface comprises a magnetic or paramagnetic particle.

98. The system or kit of claim 96 or 97, wherein the particulate support surface is coated with a member of a binding pair.

99. The system or kit of claim 96, wherein the member of the binding pair is selected from avidin, streptavidin and biotin.

100. The system or kit of claim 96 or 97, wherein the particulate support surface comprises a carboxyl or amine coating.

101. The system or kit of any of claims 96 to 100, further comprising an immobilization reagent for immobilizing a capture molecule on the particulate support surface.

102. The system or kit of claim 101, wherein the immobilization reagent is selected from avidin, streptavidin and biotin.

103. The system or kit of any of claims 95 to 102, wherein the assay surface comprises a multi-well plate.

104. The system or kit of any of claims 95 to 103, wherein the assay surface comprises a plurality of particles.

105. The system or kit of any of claims 95 to 104, wherein the assay surface comprises a plurality of discrete binding domains.

106. The system or kit of any of claims 95 to 105, comprising a targeting reagent complement immobilized on the assay surface.

107. The system or kit of any of claims 95 to 106, comprising more than one unique targeting reagent complements immobilized in discrete binding domains on the assay surface.

108. The system or kit of any of claims 95 to 105, comprising, in a separate vial, container, or compartment, and a targeting reagent complement that can be immobilized on the assay surface.

109. The system or kit of any of claims 95 to 108, comprising a component of a detection reagent.

110. The system or kit of claim 109, comprising a labeled targeting reagent.

111. The system or kit of claim 110, wherein the label comprises an electrochemiluminescent (ECL) label.

112. The system or kit of claim 111, wherein the label comprises an organometallic complex that comprises a transition metal.

113. The system or kit of claim 112, wherein the label comprises ruthenium.

114. The system or kit of claim 113, wherein the label comprises a MSD SULFO-TAG™ label.

115. The system or kit of claim 110, wherein the labeled targeting reagent comprises a primer for rolling circle amplification (RCA primer).

116. The system or kit of claim 115, further comprising a template or rolling circle amplification (RCA template).

117. The system or kit of claim 115 or 116, further comprising a detection probe that is capable of hybridizing to a rolling circle product (RCA product) generated using the RCA template.

118. The system or kit of claim 117, wherein the detection probe comprises a label.

119. The system or kit of claim 117 or 118, wherein the detection probe comprises a fluorescent label.

120. The system or kit of any of claims 95 to 119, wherein the targeting reagent comprises an oligonucleotide sequence that is complementary to an oligonucleotide sequence of a targeting reagent complement.

121. The system or kit of any of claims 95 to 120, wherein the release reagent is selected from a protease, an endopeptidase, a reducing reagent and a restriction enzyme.

122. The system or kit of claim 121, wherein the protease is selected from: a serine protease, cysteine protease, threonine protease, aspartic protease, glutamic protease, and metalloprotease.

123. The system or kit of claim 122, wherein the protease comprises proteinase K.
